(19) United States
(12) Patent Application Publication
(10) Pub. No.: US 2012/0178646 A1
(43) Pub. Date: Jul. 12, 2012

(54) HIGH THROUGHPUT METHOD AND SYSTEM FOR IN VIVO SCREENING

(75) Inventors: Herman Pieter Spaink, Oegstgeest (NL); Ronald Petronella Hubertus Dirks, Voorschoten (NL)

(73) Assignee: ZF SCREENS B.V., LEIDEN (NL)

(21) Appl. No.: 13/382,702

(22) PCT Filed: Jul. 8, 2010
(86) PCT No.: PCT/NL10/50439

§ 371 Date: Mar. 27, 2012

Related U.S. Application Data
(60) Provisional application No. 61/223,805, filed on Jul. 8, 2009.

(30) Foreign Application Priority Data
Jul. 8, 2009 (EP) 09164956.6

(51) Int. Cl. C40B 30/04; C40B 40/08; C07H 21/04; C40B 60/12

(52) U.S. Cl. 506/9; 506/39; 506/17; 536/23.1

(57) ABSTRACT

Provided is a method and system for screening chemical compounds or compositions, wherein replicating entities are introduced into the yolk of an (un)fertilized egg or embryo. The method may be extended to elucidate the mechanism-of-action of functional chemical compounds or compositions in the same method and system. The method and system may also be employed for identifying marker genes, marker proteins or marker metabolites.

### FIELD OF THE INVENTION

The present invention is in the fields of infectious diseases, evaluation of microbial probiotics, screening of pharmaceutical compound libraries, drug target identification, lower vertebrate model systems and automated high throughput screening.

### BACKGROUND

At the moment drugs screens are either performed in cell cultures or in animal models. A major drawback of cell cultures is that they are not predictive for disease symptoms in most diseases. For example, tuberculosis progression has as a hallmark the development of a granuloma which is an association of many infected host cells that cannot yet be mimicked in vitro. The major limitation of adult vertebrate test models is the small size of the population that can be economically, technically or ethically screened. At the moment the mouse model system is limited for screening only relatively low numbers of compounds. For immune related diseases, lower vertebrates have recently been shown to be highly suitable as a test model, for instance in infectious disease and cancer. Zebrafish has now been developed into a medium throughput capacity model for drugs related to immune-related diseases and cancer.

The present inventors describe a breakthrough for high throughput applications in zebrafish (Danio rerio), other cyprinid fish species (e.g. the common carp (Cyprinus carpio)) and other fish species that lay up to millions of eggs per fish that enable millions of pharmaceutical drug candidates to be tested.

For many diseases there is no fast diagnostic tool for disease progression. For instance, the formation of tuberculosis-induced granulomas in mice is very difficult to observe in the living animal and therefore post-mortem analyses are needed. This makes drug screening extremely difficult. The present inventors show a very fast high throughput test system for granuloma formation after injection of pathogens into the yolk of fish or amphibian embryos.

Thus far, a bottleneck for these applications has been the technology needed to introduce the pathogens inside the organism. The present inventors have invented methods to deliver microbial infectious agents at high throughput in such a way that it leads to disease symptoms and the expression of characteristic disease markers. This includes the description of the position and the time point of the injection and highly functional carrier materials to introduce infectious agents or tumor cells at high throughput.

Pharmaceutical drug screening is currently highly dependent on tissue culture-based screening systems. Such systems typically are capable of handling up to ten thousand compounds a day using robotic microtiter handling and pipetting systems. In principle, the limits of throughput are dependent of the speed of the analysis methods and in most cases not on the drug delivery speed.

At the multicellular organism level, one of the easiest screening systems is the nematode Caenorabditis elegans, which makes screens up to the level of cellular culture systems possible; however, these screens are only possible for compounds that can enter the organism via diffusion into the organism or via ingestion into the intestinal tract. E.g., an ingenious way to induce RNAi in nematodes is to feed them with bacteria containing dsRNA constructs. For applications in vertebrates, some compounds can also be added orally and can enter the system via the gastro-intestinal tract. It should be noted that, for vertebrate studies, the gastro-intestinal tract starts to develop at a stage where ethical regulation begins to apply. For compounds for which no such methodology is available injection methods are needed, which greatly limits the throughput level. A system has been developed for injection in Drosophila using micro-electro mechanical systems (MEMS) that can be used to perform up to 50 RNAi experiments per day (Zappe et al., 2006). In great contrast, for screening in vertebrate systems that throughput level comes far below these numbers. The state of the art is currently defined by the system of Sun, Wang and Liu (WO 2008/034249) that describes a highly accurate injection system for zebrafish embryos. This system is able to inject compounds into zebrafish embryos using automated image recognition and two micro-robots. However, in this system the throughput level is limited by the high accuracy of the injections and cannot be expected to reach levels of up to thousands embryos a day per one setup. This level is not even approaching the levels that can be reached in cellular screening systems. It should be noted that the reason for the high accuracy is the application of this injection system for the study of developmental processes influenced by the injected compounds. E.g., in the given example antisense morpholinos are tested that affect development. It is clear that any inaccuracy of the injection can lead to unwanted damage and therefore result in developmental phenotypes. In the absence of a post-screening system that can filter out phenotypes resulting from faulty injections, this throughput limitation cannot be circumvented. In summary, the current state of the art in low vertebrate screening systems can best be described as low throughput.

In the area of microbial infection or cancer xenotransplantation studies, the levels of throughput reached using injection systems in embryos are even much lower than reached by Sun et al. (WO 2008/034249). E.g., the immune effects of injected pathogenic bacteria or viruses (Levraud et al., 2007) in embryos/larvae have been described by various authors. Various species of bacteria were injected into different tissues of the embryos. These studies make use of the fact that the innate immune system of the zebrafish embryos has already developed at 27 hours post fertilization (hpf). Therefore, injection studies were performed around the onset of this developmental stage. Studies include injections into the caudal vein or the somite tissue of the tail of 27 hpf embryos (Stockhammer et al., 2009), into the hindbrain ventricle of 24-30 hpf embryos (Davis and Ramakrishnan, 2009), into the yolk or the ventral aspect of the yolk sac circulation valley of 30 hpf embryos (Prajsnar et al., 2008). In all publications, screening systems are based on either transcriptome alterations, or visually detectable phenotypes, such as granuloma formation, bacterial spread, and embryo lethality. The effect of injection with probiotic bacteria, such as lactobacillus, has not yet been evaluated. Screens have not been carried out yet at the proteomic or metabolomic level. The reported effects of the infection in embryos are mainly dependent on the strain of bacteria tested and, secondly, on the position of injection. E.g. for lethality, Prasjnar et al. (supra) showed that injection with 100 colony-forming units (cfu) of Staphylococcus aureus into the yolk of 30 hpf embryos resulted in near 100% lethality, whereas upon injection of fewer than 1200 cfu of the same strain into the ventral aspect of the yolk sac circulation valley there was 100% survival at 48 hpf. Until now, all reported microbial injection studies in zebrafish embryos have been accomplished manually. Furthermore, no screens for drugs that influence the infection process in embryos have been reported yet. The same is true for screening of drugs against xenotransplanted cancer cells for which methods have been published of injection in the yolk after 3.5 hours post fertilization (Lee et al. 2005). Intrayolk injections of embryos at later developmental stages has the following disadvantages: (1) a gradual decrease in the yolk to embryo ratio makes automatic injection increasingly difficult; (2) this results in an increasing chance of damaging the embryo proper during the injection; (3) the injected biosystems will be asymmetrically divided over the different parts of the developing embryos.

Early yolk infections with viruses have been mentioned as useful models for testing possible therapies (WO 2009/056961); however, this prior art does not show replicability of the viral particles or survival of the embryos, nor is any high throughput method suggested to perform screening. Prior art articles to this work (Levraud et al. (2009) Infection and Immunity 77 (9), 3651-3660; van der Sar et al. (2003) Cellular Microbiology 5 (9), 601-611) show that zebrafish embryos injected with replicating organisms at 24 hours post fertilization and embryos injected at later stages do not survive this treatment for longer than two days. According to Van der Sar et al: “The yolk of S. typhymurium may be used as an in vivo growth control for bacterial mutants. The infected zebrafish embryos survived the Ra mutant infection of the yolk for two days. At that time the yolk did not contain the bacteria, which entered the embryo itself and rapidly killed it.” Levraud et al. reported: “We tested alternative ways to infect the larvae: injections performed directly inside the yolk cells (54 hours post fertilization) resulted in death faster and at lower doses than i.v. injections; in fact, L. innocua readily killed zebrafish larvae under this condition”

Large scale comparisons of the effect of microbial or cancer cell agents have not been performed yet. For large scale drug screens and microbial and cancer cell comparison screens, automation of the injection procedure would be highly desirable. It is at the moment not yet possible to automate injection of microbes and cancer cells under our definition of high throughput. It might be possible in due course to use the automation system of Sun et al. (supra) for injection of bacteria, cancer cells or viruses in the above described stages and positions of the embryo, although this has not yet been described and would mean that several technical problems would have to be solved. In any case, the throughput level cannot be expected to become higher than described for the morpholino injections in early embryos.

In view of the foregoing, there is a need in the art for a high throughput system and method which is able to inject microbes and large substances into vertebrate embryos. Such system and method should lead to a detectable response, e.g., at the visual, or transcriptome, proteome or metabolome level in such a way that the readouts have relevance for drug screens in infection studies. Such a system should also be adaptable for the screening of microbial characteristics needed for virulence or probiotic properties. If a system like this would be available, fish embryos would become a highly desirable immune biosensor model system that would be applicable in many high throughput assays in the biomedical and microbial food industry.

### SUMMARY OF THE INVENTION

The present inventors explored the possibilities to design a high throughput system and method that relies only on fast injection and neglects accuracy of injection, which system and method may be combined with post-injection high throughput filtering for embryos that were not injected in a desired way. Such approach has not previously been reported in alternative injection systems for vertebrate embryos. Instead of accurately injecting embryos with a capacity of up to thousands per day per system, the present inventors explored the possibility of inaccurately injecting embryos with a capacity of up to ten thousands per day per system, optionally combined with high throughput post-screening for accuracy.

In a first aspect, the present invention relates to a method for screening chemical compounds or compositions in an embryo or larvae system, comprising the steps of:

providing a plurality of start biosystems, said start biosystems being selected from living eggs or embryos of aquatic developing chordates, and said start biosystems being in the stage prior to 22 hours post fertilization;

introducing one or more replicating entities in the yolk of at least a set of said start biosystems;

exposing a set of said start biosystems to said chemical compounds or compositions;

allowing said start biosystems to develop to result in a plurality of embryos or larvae;

determining a response in said embryos or larvae, and

correlating said chemical compounds or compositions and said response.

In another aspect, the present invention pertains to method for determining a mechanism underlying the effect of functional chemical compounds or compositions on disease development in an embryo or larvae system, comprising the steps of:

providing a plurality of start biosystems, said start biosystems being selected from living eggs or embryos of aquatic developing chordates, and said start biosystems being in the stage prior to 22 hours post fertilization;

introducing one or more replicating entities capable of effecting disease development in the yolk of at least a set of said start biosystems;

exposing said set of said start biosystems to said functional chemical compounds or compositions;

exposing at least a subset of said start biosystems to one or more gene-function-modifying molecules;

allowing said start biosystems to develop to result in a plurality of embryos or larvae;

determining a response in said embryos or larvae,

correlating said gene-function-modifying molecules and said response, and

identifying gene-function-modifying molecules counteracting the effect of said functional chemical compounds or compositions on disease development.

In a further aspect, the present invention provides a high throughput screening system for a set of chemical compounds or compositions using a plurality of start biosystems having a yolk, said start biosystems selected from the group consisting of living eggs and living embryos of aquatic developing chordates, said system comprising:

a controller;

a transporter, operationally coupled to said controller, for passing start biosystems individually past an introduction position;

an injector, operationally coupled to said controller, adapted for intrayolk introduction of at least one living entity in at least a set of said start biosystems at said introduction position;

an exposure system for exposing at least a set of said start biosystems to one or more of said chemical compounds or compositions, said exposure system operationally coupled to said controller;

a first detector, operationally coupled to said controller, for measuring a first response of said each of said start biosystems and transmitting the measurements to said controller, said controller storing said measurements coupled to the replicating entity introduced into a biosystem and the chemical compound or composition that biosystem was exposed to.

In another aspect, the present invention is concerned with a method for identifying marker genes, marker proteins or marker metabolites characteristic for a specific disease or situation, said method comprising the steps of:

providing a plurality of start biosystems, said start biosystems selected from living eggs or embryos of aquatic developing chordates, and said start biosystems being in the stage prior to 22 hours post fertilization;

introducing one or more replicating entities capable of effecting said specific disease or situation in the yolk of at least a set of said start biosystems;

determining a transcriptome, proteome or metabolome in at least said working set of biosystems;

comparing the transcriptome, proteome or metabolome of biosystems in which replicating entities have been introduced with the transcriptome, proteome, or metabolome in biosystems in which no replicating entities have been introduced; and

identifying marker genes, marker proteins or marker metabolites for said specific disease or situation.

In a final aspect, the present invention provides the use of a living embryo or larvae of an aquatic developing chordate having a replicating entity capable of effecting a disease introduced in its yolk for screening the effect of a chemical compound or composition on said disease.

### BRIEF DESCRIPTION OF THE DRAWINGS

The invention will be further elucidated referring embodiments of a high throughput screening system shown in the attached drawings, wherein:

FIG. 1 illustrates a schematic flowchart of a high throughput automated compound library screening method based on intrayolk injection of fish embryos;

FIG. 2 illustrates a schematic overview of an automatic high throughput device used for intrayolk injection using a plate with spaced containers and an injector;

FIG. 3 illustrates an overview of the embryonic fish stages used in the method;

FIG. 4A illustrates an embryo holding device of the type “array plate” in top view, FIG. 4B shows left part of an embryo holding slide and right part of a top or bottom slide, and FIG. 4C shows the embryo holding device in cross section;

FIG. 5A illustrates an embryo holding device of a transporter of the type “half open tube” in transverse cross section, in FIG. 5B part of the holding device in top view and in FIG. 5C in top view large part of the transporter;

FIG. 6 illustrates an embryo holding device of the type “continuous flow carousel”, and FIG. 6A a holding cavity in cross section;

FIG. 7 illustrates a transporter comprising an embryo holding device with an oval capillary that allows hatched embryos (˜2 dpf and older) to flow through in only four possible orientations. This allows the intrayolk injection of all embryos via a central hole, located perpendicular to the flow direction;

FIG. 7A shows the capillary in cross section as indicated in FIG. 7.

FIG. 8A illustrates the COPAS XL Biosorter profile of a zebrafish embryo after intrayolk injection with CherryRed-labeled Mycobacterium marinum, and FIG. 8B a picture showing laminating mycobacteria in an embryo;

FIG. 9 shows selected marker genes showing specific expression changes upon infection with either BCG (Bacille Calmette Guerin (BCG) vaccine for tuberculosis, which containes a live attenuated (weakened) strain of Mycobacterium bovis), Rhizobium, Lactobacillus casei shirota (“Yakult”), Trypanosomes, Mycobacterium leprae, Mycobacterium smegmatis, and Mycobacterium marinum.

### DETAILED DESCRIPTION OF THE INVENTION

### Method for Screening Chemical Compounds or Compositions

The present invention provides for a method for screening chemical compounds or compositions in an embryo or larvae system, comprising the steps of:

providing a plurality of start biosystems, said start biosystems being selected from living eggs or embryos of aquatic developing chordates, and said start biosystems being in the stage prior to 22 hours post fertilization;

introducing one or more replicating entities in the yolk of at least a set of said start biosystems;

exposing a set of said start biosystems to said chemical compounds or compositions;

allowing said start biosystems to develop to result in a plurality of embryos or larvae;

determining a response in said embryos or larvae, and

correlating said chemical compounds or compositions and said response.

It was surprisingly found by the present inventors that injection of replicating entities into the yolk of start biosystems that are at earlier stages (prior to 22 hours post fertilization), which has not been attempted previously, is not lethal up to at least 5 days post fertilization. This is the first time that any analysis of yolk-infected embryos is possible at this stage after fertilization (5 days post fertilization). Previously, yolk-injected embryos did not survive intra-yolk injection past two days post fertilization.

The process of finding a new drug against a chosen target for a particular disease usually involves high-throughput screening (HTS), wherein large libraries of chemical compounds or compositions are tested for their ability to modify the process under investigation. In the method of the present invention, chemical compounds or compositions are screened for their capability of counteracting or preventing development of a certain disease or condition which can be effected by said one or more replicating entities, e.g., bacteria, protists, and the like.

As used herein, the term “chemical compounds or compositions” refers to any compound or combination of compounds, including a compound incorporated in a certain matrix (composition). The matrix may be an aqueous solution, or an organic solvent, or any other matrix. The term “chemical compounds or compositions” as used herein includes, without limitation, inorganic compounds, organic compounds, protein compounds, vaccines, and the like.

An embryo is a multicellular eukaryote in its earliest stage of development from, the time of first cell division until birth, hatching, or germination.

A larvae is a young (juvenile) form of an animal with indirect development, going through or undergoing metamorphosis (for example, insects, amphibians, or cnidarians).

In a step of the method of the invention, a plurality of start biosystems are provided, said start biosystems being selected from living eggs or embryos of aquatic developing chordates. The start biosystems may be selected from living eggs or embryos. The term “eggs” as herein used refers to an unfertilized egg as well as a zygote, resulting from fertilization of the egg.

The eggs or embryos may be derived from any animal, but are preferably derived from aquatic developing chordates. As used herein, the term “aquatic developing chordates” refers to chordates laying eggs, which eggs are fertilized outside the chordate's body, and which fertilized eggs further develop outside the chordate's body. In an embodiment, the eggs or embryos are soft-shelled.

The eggs may be unfertilized or fertilized, the latter herein also being referred to as “zygotes”. The embryos may be in any stage of development, e.g., the earliest stages of development, i.e. the 1-16 cell stage of development, the blastula stadium, and the like. The start biosystems are preferably in the stage prior to 22 hours post fertilization. In an embodiment, the yolk is relatively large relative to the total size of said egg or embryo. The substance may also be introduced into the yolk of embryos at later stages of development, from sphere stage until just after hatching stage (approximately 3 dpf (days post-fertilization). This embodiment may advantageously be used for biological validation of data obtained with earlier stage embryos to rule out abnormal development shortly after introduction of the replicating entities.

It is preferred that the embryos are lower vertebrate embryos, or mutant or transgenic embryos thereof. These embryos include, without limitation, embryos of the zebrafish, common carp, other cyprinids, other culturable fish species which lay many eggs and can be used for in vivo and in vitro fertilization, amphibian species, zebrafish transparent mutants (casper), and transgenic carps. Fish eggs may be fertilized using standard procedures well known in the art. The most common reproductive strategy for fish is known as oviparity, in which the female lays undeveloped eggs that are externally fertilized by a male. Typically large numbers of eggs are laid at one time and the eggs are then left to develop without parental care.

The present inventors have recently demonstrated that the method of the invention can also be performed using embryos of pre-vertebrates such as sea squirts as start biosystems. Surprisingly, Mycobacterium marinum was also detectable at least one day after injection of the embryos. The advantage of using pre-vertebrates is that they do not fall under any regulation on animal experimentation in any country. The genome of the sea squirt is known and contains many immune genes which are related to the immune genes in vertebrates. Examples are the Toll-like receptors. Immune screening in sea squirts and other pre-vertebrates with Toll-like receptors may be relevant for biomedical applications. Thus, the use of living eggs or embryos of pre-vertebrates, e.g., sea squirts, is also included in the methods of the present invention.

It is expected that every fish species will be suitable for the method of the invention. The method of the invention may also be applicable to any other organism that produces externally fertilized eggs, such as frogs. In many of the experiments set forth below use was made of the zebrafish embryo as a versatile model for testing the effect of introducing replicating entities into the yolk. In order to follow these entities after introduction use may be made of transgenic zebrafish. These zebrafish may express a gene for an autofluorescent protein under control of a tissue specific promoter. For instance, in the experiments set forth below use was made of the fli-1 GFP line as constructed by Lawson and Weinstein (2002) in order to follow spread of the entities into the blood vessels. Another example that may be employed is the MPO-GFP line constructed by Renshaw et al., (2006) and the MYCH-YFP line constructed by Meijer et al (2008) in order to visualize immune cells such as neutrophils and granuloma structures in a living embryo. These autofluorescent proteins may be monitored simultaneously with the introduced replicating entities, which may have been labeled with a different fluorescent marker, using fluorescence detection methods described below. In this way it may be possible to monitor whether replicating entities such as bacteria disseminate in the blood or are taken up by immune cells and enter granuloma structures. Introduced replicating entities may also be stained by fluorescent markers that are sensitive for degradation or low pH in the lysosomes. In this way the disappearance of fluorescence is a read-out for the digestion of replicating entities by phagosomes. Such technologies are not restricted to zebrafish only. It is possible using standard techniques to make transgenics in all other fish species, as exemplified in medaka or salmon (Takagi et al., 1994; Fletcher et al., 2004). In addition to using transgenics it may also be useful to make use of mutant fish species. A useful example is the use of transparent mutants of the zebrafish, for instance nacre, roy or casper (White et al, 2008). Several published albino mutants of zebrafish (White et al., 2008) were used in the method of the invention and absence of pigmentation was shown to be an advantage for purposes of high throughput screening. By crossing albino mutants with transgenic lines albino-fluorescent offspring can be obtained that are highly useful for fluorescence screening of introduced replicating entities. The zebrafish also offers the availability of various immune mutants. The use of such immune mutants may allow testing the role of the immune system in progression of disease symptoms. It may also allow performing follow-up studies of the action of pharmaceutical drug candidates that have been identified using the method of the invention. For instance, a mutant in the TLR (toll like receptor) pathway may be used to test whether particular pharmaceutical drug candidates that are active against tuberculosis are functioning via this pathway. Mutants in gut or mouth development may be used to test whether pharmaceutical drug candidates are active by entrance into the intestinal system. Mutants in blood vessel formation may be useful to test whether introduced replicating entities are spread via the blood vessel system (an example of the latter application was recently published by Marques et al., 2009).

For many purposes fish species are highly useful for high throughput screening purposes. For example, the common carp is highly related to zebrafish and it has been shown by the present inventors that it can be employed using the method of the invention. Other fish that are easy to culture and provide a large number of offspring such as tilapia and pike-perch are also amenable to the method of the invention. The present inventors have shown that after injection of Mycobacteria in the yolk, granulomas are formed in various other parts of the body. The advantage of carp fish is that every female fish is capable of producing up to a few hundred thousands eggs and that these can be efficiently fertilized in vitro (http://www.fishbase.org/summary/SpeciesSummary.php?id=1450). In addition to the advantage of numbers, carp fish offers another advantage: the genomic homogeneity of the eggs is easier to control than is the case for fish such as zebrafish that provide small clutches of 150 to 200 eggs. Thus, for zebrafish a large number of parent animals is required to obtain the high numbers of eggs or embryos needed for high throughput screening and it is currently difficult to obtain genetically homogeneous parent populations of zebrafish or other small aquarium fishes, due to difficulties of inbreeding. In contrast one clutch of eggs from a common carp of hundred thousand eggs can all be fertilized by the same parent and therefore the genetic diversity is less. This advantage may be further improved by using double haploid carps that may be obtained by androgenesis or alternatively gynogenesis, techniques that are well established for various fish species (e.g. Paschos et al., 2001). Common carp was shown to be suitable in the method of the present invention.

A disadvantage of carp eggs or embryos is that the eggs have the tendency to stick together. When this is undesired, this may be prevented by adding compounds externally to the medium comprising the eggs or embryos. Non-limiting examples of such compounds include pineapple juice (That et al., 2004), salt/urea/tannin (Cabrita et al., 2009), or cow's milk (Recoubratsky et al., 1992). However, in some embodiments, it may be advantageous to have the eggs sticking together. This is particularly the case when one wishes to have the eggs positioned in a thin regular layer allowing injection directly on the thin layer of eggs. Regularity of the layer may be imposed by using a raster that is pressed on the eggs just before fertilization. The openings of the raster may hold the eggs in place at a regular distance. Subsequent fertilization through the raster may then lead to a regularly spaced matrix of eggs that can be automatically injected based on the spacing of the raster used to align the eggs.

In case sticking of carp eggs or embryos is not favored, it is also possible to use relatives of the carp that produce non-sticking eggs. For particular experiments eggs of other fish species may also have advantages. Examples of these are fish species that grow at higher temperatures which will enable screening of replicating entities that are temperature sensitive, such as microbes or cancer cells that do not grow at temperatures lower than 37 degrees. In this case many fish species that produce a large numbers of eggs and also grow at these temperatures will be highly suitable. For instance, such fish species include, without limitation, tropical carp species and gourami species.

In contrast, for replicating entities that can not withstand relatively high temperatures, it may be beneficial to use fish species that grow at low temperatures, like salmonids. It may also be advantageous to use fish species that spawn in salt water, e.g., upon testing microbial strain that normally infects salt water fish. Also, in some test facilities salt water is available at large quantities and the use of salt water fish could then be economically highly favorable. Many salt water fish can now be propagated in captivity and therefore extremely large supplies of eggs, fertilized eggs and embryos for these fish species are easily available. Non-limiting examples of such fish species include the family of Scophthalmidae (e.g. turbot). For some applications in pharmaceutical screening it may be beneficial to use the same parent animal for producing offspring to limit variations in results due to genetic variations. In this case use can be made of fish species that are able to reproduce for many years, such as koi carp or goldfish that are know to be able to spawn for decades.

Any number of start biosystems may be provided. The method of the invention is suitable for high throughput purposes, but may also be employed for non-high throughput purposes. In an embodiment, at least about 96, at least about 150, at least about 200, at least about 300, at least about 400, at least about 500, at least about 600, at least about 700, at least about 800, at least about 900, or at least about 1,000 start biosystems may be provided.

In a further step of the method of the invention, one or more replicating entities are introduced into the yolk of at least a set of said start biosystems. The term “replicating entities” as used herein refers to living entities as well as viruses, and includes, without limitation, bacteria, fungi, yeasts, protists, cancer cells, clusters of cancer cells, viruses, and any combination of these. In an embodiment, said replicating entities are capable of effecting or causing a disease, condition, or situation. For example, said replicating entities may be pathogens, or probiotic microbes.

The term “microbes”, as used herein, refers to both prokaryotic and eukaryotic microorganisms, and includes bacteria, archaebacteria, yeasts, and fungi. The cancer cells referred to may be any type of cancer cells such as from human, rodent and fish. The term includes cells from cancer cell lines and immortalized cancer cell lines. The term “unicellular eukaryotic organisms” as used herein refers to any unicellular organism, and includes protists (such as Plasmodia, e.g. Plasmodium falciparum, P. berghei); Trypanososomes, (e.g. Trypanosoma brucei, T. carassii; Leishmania species), eggs of nematodes and trematodes (such as Schistosoma).

Non-limiting examples of replicating entities selected from the group of bacteria, archaebacteria, yeasts, fungi, cancer cells, viruses, and protists include: granuloma-inducing mycobacteria (e.g. Mycobacterium marinum, M. tuberculosis), non-granuloma-inducing mycobacteria (Mycobacterium smegmatis, M. bovis), neuron-infecting mycobacteria (e.g. M. leprae), pathogenic gram-negative bacteria (e.g. Edwardsiella tarda and Salmonella species), pathogenic gram-positive bacteria (e.g. Streptococcus iniae), non-pathogenic gram-positive bacteria (e.g. Bacillus subtilis), and lactobacilli, such as Lactobacillus casei shirota, L. casei defensis, L. casei rhamnosus), non-pathogenic gram-negative bacteria (e.g. Rhizobium leguminosarum and Agrobacterium tumefaciens), non-pathogenic yeasts (e.g. Saccharomyces cerevisiae), pathogenic yeasts (e.g. Candida albicans), non-pathogenic fungi (e.g. Penicillium camemberti, P. candidum), pathogenic fungi (e.g. Aspergillus fumigates, A. niger), protists (such as Plasmodia, e.g. Plasmodium falciparum, P. berghei); Trypanososomes, (e.g. Trypanosoma brucei, T. carassii; Leishmania species), eggs of nematodes and trematodes (such as Schistosoma), viruses (e.g. spring viremia of carp virus (SVCV)), vertebrate cancer cells such as from human, rodent and fish, the causative agent of Lyme disease, specifically bacteria from the genus Borrelia. It also envisaged that prions or organelles of microorganisms may be introduced. A comparison of e.g. Mycoplasma and cancer cells separately injected or co-injected may predict the presence of Mycoplasma infections in cancer cell cultures.

The number of replicating entities that are introduced depend on various factors, including the type of replicating entity to be introduced. Generally speaking at least one replicating entity should be introduced. The maximum amount of replicating entities to be introduced depends largely on the rate of replication of said replicating entity or entities. For replicating entities that replicate fast, a smaller number of replicating entities should be taken compared to replicating entities that replicate slowly. For fast-replicating replicating entities, lysis of the yolk may be prevented by selecting a small number of replicating entities. For slow-replicating replicating entities, as many replicating entities as possible may be introduced. The correct number still acceptable in the method of the invention can easily be determined by the skilled person by introducing a variety of concentrations of said replicating entities in the method of the invention and determining the concentration at which the start biosystems remain intact, and a response can be observed. The maximum number of replicating entities may be dictated by the maximum volume that may be introduced into the yolk. As a rule of thumb, for zebrafish embryos a volume of about 25% of the volume of the yolk may be introduced into said yolk. However, this may vary depending on the start biosystem employed.

The replicating entities may be combined with a carrier material prior to introduction into the start biosystems. A “carrier material” as referred to herein refers to a non-immunogenic polymer or matrix, which is inert and does not chemically react with chemical compounds or compositions. Such carrier material includes, but is not limited to, polyvinylpyrrolidone (PVP)(for example, PVP-40K or PVP-200K), Matrigel, polyethyleneglycol (PEG; e.g. PEG-6000), dextran (e.g. Dextran-40K) or Ficoll (e.g. Ficoll-400). In one embodiment of the invention, the replicating entities may be injected with needles. In this case, the substance may be re-suspended in highly viscous solutions of polymers, e.g. polyvinylpyrrolidone (PVP)(for example, PVP-40K or PVP-200K), Matrigel, polyethyleneglycol (PEG; e.g. PEG-6000), dextran (e.g. Dextran-40K) or Ficoll (e.g. Ficoll-400). In case the polymer used as carrier material is PVP, it is preferred that the PVP is used in an amount of about 0.25 to about 5% (w/w), such as about 0.5% to about 4.5% (w/w), about 1 to about 4% (w/w), about 1 to about 3% (w/w), about 1.5 to about 2.5% (w/w), or about 2% (w/w). The concentration of carrier material will generally depend on the type of carrier material, the intended application, and whether further compounds are to be introduced. For example, if gene-silencing compounds are to be co-injected with the replicating entities, the concentrations of carrier material, e.g. PVP, will have to be limited in order to allow the gene-silencing compounds to reach their gene targets. In such case, a relatively low concentration of about 0.5% PVP may be useful. In contrast, if hydrophobic molecules are to be included with the carrier material, relatively high concentrations of PVP may be used. The PVP may be further mixed with cyclodextrans. In an embodiment the carrier material-comprising solution may further comprise a buffer to maintain the pH at a range of about 5-9, preferably about 6-8. The carrier material allows for slow diffusion of the replicating entities into the yolk, thereby avoiding a burst of the replicating entities in the yolk, and subsequent consequences of lethality for the start biosystem. This is particularly the case for up to the 16-cell stage, as the embryonic cells are not yet completely separated from the yolk. Particularly at these stages, the carrier material inhibits the rapid entry of the replicating entities from the yolk into the open cells of the embryos.

The step of introducing said one or more replicating entities may take place using any method and means known in the art, e.g., by injection.

Injection may take place by any means known in the art. The injection means may e.g. be in the form of a micropipette having a sharp tip (e.g., glass capillary or microfabricated needle). For zebrafish embryo injection, the size of zebrafish embryos requires microneedles with a tip length of about 600-2000 μm and outer diameter of 5-100 μm throughout the 600 μm length. For zebrafish embryo injection, the injection needles also should be strong enough without buckling under hundreds of microNewton penetration forces. One skilled in the art will be capable of determining the correct injection means depending on the type of start biosystem that is employed in the method of the present invention and the stage (size) of said start biosystem that is to be injected.

Injection may be performed using glass needles which comprise suspensions of the one or more replicating entities, and optionally said chemical compounds or compositions and/or further molecules, that are delivered into the yolk of said start biosystems, e.g., using pressure. Injection may be accomplished via simple repetitive and coordinated computer control of a stage positioner, micromanipulator and pressure unit. Alternatively, the one or more replicating entities may be injected using ballistic bombardment (also called ballistic delivery).

The replicating entities may be formulated together with a carrier material, as described hereinabove. In case the substance is injected with needles, the substance may be suspended in highly viscous solutions of polymers, e.g. polyvinylpyrrolidone (PVP), Matrigel, polyethyleneglycol (PEG) or Ficoll. In case the substance is injected using ballistic bombardment, it may be embedded in a matrix of non-immunogenic solid carrier material, such as cellulose-sulfate or plastic. For application with microbes as a substance, it may be beneficial to use degradable material. The degradability is either achieved by enzymes of the start biosystems (biodegradation) or by external treatment of the start biosystems with a trigger, such as light, that degrades the carrier material.

In another step of the method of the invention, a set of said start biosystems is exposed to said chemical compounds or compositions. The exposing step may take place externally, i.e., said chemical compounds or compositions are added externally to said egg or embryo. However, there is often no knowledge on the properties of the drugs with regard to penetration in tissues and cell, and chemical compounds and compositions may demonstrate no effect solely due to penetration issues. The exposing step may also take place by internally introducing said chemical compounds or compositions, e.g. by injection thereof. The chemical compounds or compositions may be introduced prior to, simultaneously with, or after the introduction of the one or more replicating entities. In case the chemical compounds or compositions are introduced simultaneously with said one or more replicating entities, they chemical compounds or composition and the replicating entities may be co-administered, e.g. by means of co-injection. In order to obtain information regarding the penetrating power of the chemical compounds or compositions into start biosystems, one may select a first set of start biosystems to be exposed to said chemical compounds or compositions externally, and select a second set of start biosystems to be exposed to said chemical compounds or compositions internally, and compare the effect the exposure sorts.

In an embodiment, it is registered which of said embryos was exposed to which of said chemical compounds or compositions. Typically, the chemical compounds may be applied to subsets of the injected embryos, e.g., in microplate format. Commercially available state of the art pipetting robots, e.g. Hamilton, keep a register of what is pipetted in which well. Chemical compounds or compositions are preferably incorporated in a solvent that is not harmful to the embryos. Non-limiting examples of solvents that can be used are water, aqueous solutions of cyclodextrans, or low concentrations of DMSO in water.

In another step of the method of the invention, said start biosystems are allowed to develop under optimal conditions (oxygen and temperature) for said biosystems and said replicating entities, to result in a plurality of embryos or larvae. Subsequently, a response is determined in said embryos or larvae. The response may be any response that can be detected in said embryos or larvae. Such response includes, without limitation, responses on a physical level, transcriptome level, proteome level, metabolome level, and the like. Responses on a physical level include optical responses, paramagnetic responses, and the like. A non-limiting example of an optical response is the microscopic screening for granulomas in embryos or larvae of fish after injection of eggs or embryos with Mycobacterium tuberculosis. The response of the start biosystems to the intrayolk injection of microbes or cancer cells may be tested in the presence or absence of chemical compounds or compositions. Several assays have been developed for various fields of applications. In an embodiment, known genetic or proteomic immune markers may be used, or novel markers discovered based upon the method of the present invention may be used. Novel markers may be discovered by comparing the transcriptome, proteome, metabolome, or epigenetic responses of start biosystems in which one or more replicating entities are introduced with the transcriptome, proteome, metabolome, or epigenetic responses of start biosystems which have followed the exact same procedure with the exception of the introduction of said one or more replicating entities.

From total genome based data sets subsets of markers can be defined that, because of their lower complexity, may be easier to apply in high throughput screening. These markers may be read out by DNA-based assays, such as PCR and restriction enzyme analysis, RNA-based assays, such as RT-PCR, RT-MLPA, or promoter-based transgenic fluorescent or luminescent reporter constructs, antibodies (e.g. ELISA), and sensors for particular metabolic compounds. Microscopic screening may be applied to visualize disease-related phenotypes. This varies for different types of microbes or cancer cells. It is not difficult to screen at high throughput for the effect of intrayolk injection of microbes or cancer cells on viability of the embryos. Optimal time points have been established for measurements for each of the above mentioned microbes or cancer cells. The maximum time at which scoring took place was determined by ethical regulations in the country in which the tests were performed. E.g., in most European countries, this time point is limited to approximately 5 dpf. Using this test system, the effect of a pharmaceutical drug candidate may be evaluated by its diminishing effect on lethality. Likewise, the positive effect of a probiotic may be scored by its diminishing effect on lethality, when injected in a mixture with pathogenic microbes. E.g. in the case of granuloma-inducing bacteria, the granulomas may be visualized by using fluorescent of luminescent bacteria and/or transgenic fish in which immune cells are labeled by fluorescence of luminescence. It has been demonstrated that the injection of mycobacteria into the yolk of fish embryos using the method of the invention leads to the reproducible formation of granulomas at 3-5 dpf that can be detected at a high throughput level. Since granulomas are the hallmark of tuberculosis, this enables screening at a high throughput level for drugs against tuberculosis. As a proof of concept it has been shown that a known anti-tuberculosis drug was successful.

In another step of the method of the invention, said chemical compounds or compositions and said response are correlated. Thus, the effect of said chemical compounds or compositions on a disease or condition effected by the one or more replicating entities may be established. The method of the invention is particularly suitable for identifying chemical compounds or compositions that may be useful in preventing and/or treating a disease or condition caused by the one or more replicating entities. Alternatively, the method of the invention may be suitable for identifying chemical compounds or compositions boosting a positive effect of said one or more replicating entities, particularly in case of beneficial replicating entities such as probiotic microbes which may improve the general condition of said start biosystems. The registration of which of the start biosystems were exposed to which chemical compound or composition is matched with the response that has been determined for each of the embryo or larvae developing from said start biosystem. Thus, it can be determined which chemical compound or composition is capable of establishing a certain desired response.

For example, the introduction of Mycobacterium tuberculosis generally leads to the formation of granulomas which is a hallmark of tuberculosis. However, in the presence of a certain chemical compound (e.g., ETB067) granuloma formation does not occur. Thus, ETB067 inhibits granuloma formation by Mycobacterium tuberculosis and may be a pharmaceutical drug candidate for prevention and/or treatment of tuberculosis.

The method of the invention allows combining one or more disease factors (herein also referred to as “pathogens”), probiotics, and/or chemical compounds or compositions in a single injection, without affecting the throughput level of the screening. In addition, it is highly sensitive and discriminative. Using the method of the present invention, it is possible to screen for the effect of pharmaceutical candidate drugs against particular phenotype associated with infectious diseases or cancer progression. Furthermore, the test system may be used to identify possible pathogenic contaminants in materials and give a rapid readout of their potential risk factor. Such materials may be biomaterials, such as food samples, or medical implants. For example, Staphylococs (especially Staphylococcuc aureus, Staphylococcus epidermidis, Staphylococcus carnosus) and Cryptococs (especially Cryptococcus neoformans) are often found as infection attached to medical implants. It is commercially interesting to test the combination of various implant materials with the microbes such as these mentioned. The implant materials are different types of plastics or metals. Comparison is made with our standard carrier materials, such as PVP. The latter may require that small fractions of the material can be sampled. Until now, the method of the invention has been validated in zebrafish and carp embryos, but the methods can be readily extended to screening in other fish or amphibian embryos, eggs or zygotes.

In an embodiment, a working set of start biosystems or embryos or larvae may be selected prior to at least the correlation step. In the working set of start biosystems or embryos or larvae those start biosystems or embryos or larvae that are either incorrectly injected or suffered developmental defects not related to immunity, are filtered out.

This prescreen advantageously does not harm viability of the start biosystems, embryos, or larvae. In one embodiment of the method of the invention, the selection step is based on light detection, e.g., using prior art technology, such as the COPAS Biosorter from Union Biometrica. In an embodiment, the selection step may be accomplished by employing transgenic embryos with internal fluorescent or luminescent indicators of viability and/or developmental stage in the method of the invention. Usually, the selection step is not required as start biosystems have been incorrectly injected, do not develop to the same stage as properly injected start biosystems.

In an embodiment, the start biosystems are in the stage of up to the blastula level (up to 128 cells). In another embodiment, the start biosystems are in the stage of up to the morula level (up to 16 cells). In yet another embodiment, the start biosystems are in the stage of the zygote level (fertilized egg). In another embodiment, the start biosystems are embryos of aquatic developing chordates.

Said one or more replicating entities may be selected from the group consisting of bacteria, fungi, yeasts, protists, and combinations thereof.

In another embodiment, the one or more replicating entities comprise cancer cells, or clusters of cancer cells.

In a further embodiment, said one or more replicating entities comprise viruses.

In an embodiment, said one or more replicating entities are comprised in a volume of below about 3 nanoliters, in an embodiment below about 2 nanoliters. Such volume approximates the maximum volume that may be injected into the yolk of fish eggs or embryos.

In an embodiment, said introducing of said one or more replicating entities comprises injecting said replicating entities in said yolk. Said injecting may comprise injection via a needle or using ballistic delivery.

In an embodiment, said exposing to said chemical compounds or compositions comprises introducing said chemical compounds or compositions into the yolk. The exposing step may be performed simultaneously with said introduction of said one or more replicating entities, or after introduction of said one or more replicating entities. Alternatively, said exposing step may be performed prior to introduction of said one or more replicating entities.

In an embodiment, the start biosystems may be mounted at high density in a carrier device (or holder) at regular spacing. Cover slides may be used that keep the start biosystems in the holder during subsequent steps, in particular injection. In case of sticky eggs, e.g. carp eggs, are employed, start biosystems may be held in place via their own capacity to stick to materials. The carrier device or holder may be made of any material, for example of metal, plastic, ceramic or glass.

In an embodiment of the invention, the carrier device (or holder) may be a plate with more than about 96, about 150, about 200, about 300, about 400, about 500, about 600, about 700, about 800, about 900, or about 1,000 regularly spaced holes, each of which can hold a single start biosystem. Start biosystems may be prevented from leaving the holes by a cover slide which contains smaller injection holes.

In another embodiment, the carrier device may be a semi-open hollow tube in which start biosystems are situated side by side resulting in a regular spacing. Start biosystems may be prevented from leaving said hollow tube by a cover slide with a slit.

In yet another embodiment, the carrier device may be a flow-through system, consisting of a rotating disc which has the capacity to incorporate single start biosystems in regularly spaced holes. In this case, start biosystems may be held in place by a build-in device that applies fluctuating pressure and underpressure.

In another embodiment, the method of the invention is applied for determining a mechanism underlying an effect established by said chemical compounds or compositions, said method further comprising the step of introducing one or more gene-function-modifying molecules in the yolk of at least a set of said start biosystems.

The method of the invention allows combining one or more disease factors (herein also referred to as “pathogens”), probiotics, and/or chemical compounds or compositions in a single injection, without affecting the throughput level of the screening. As set forth hereinabove, by testing chemical compounds or compositions both internally and externally, the whole organism permeability properties towards the chemical compounds or compositions can be assessed.

In the past, many pharmaceutical screening methods were carried out using libraries of chemical compounds or compositions, which libraries had been developed against a particular target inside cells or organisms. However, interesting putative pharmaceutical drug candidates are available of which no target is known, e.g., many natural compounds (for example, Chinese herbs that have proven effects on health but are not approved as medicinal treatments by the FDA). In such cases, the method of the invention can be highly beneficial.

For example, the method of the present invention allows combining a replicating entity, a chemical compound or composition, and a gene-function modifying molecule. The outcome of the host response in the experiment therefore is dependent on three factors: 1) the replicating entity, 2) the chemical compound or composition and 3) the gene-function-modifying molecule.

For example, the injection of a certain microbe (e.g., Mycobacterium tuberculosis) will lead to the formation of granulomas which is a hallmark of tuberculosis. However, in the presence of a certain drug (e.g. ETB067) the granuloma formation is not occurring. By discovering this pharmaceutical drug candidate an important new question arises: how does it function? By co-injecting members of a library of gene-function-modifying molecules it can be tested whether the drug is still functional in the presence of a specific gene-function-modifying molecule. If the presence of a gene-function-modifying molecule will prevent the effect of the drug, this means that the function of the drug is dependent on the gene the function of which was affected by the gene-function-modifying molecule. Gene-function-modifying molecules that have been shown to be useful for this purpose in fish and frog embryo are called morpholinos.

Even though very high throughput levels may be achieved using the method of the present invention, it is envisaged that a pre-screen is performed for chemical compounds or composition affecting the host response when only replicating entity has been introduced. Any compounds or compositions affecting said host response may then be further tested with gene-function-modifying molecules.

Thus, in a further aspect the present invention relates to a method for determining a mechanism underlying the effect of functional chemical compounds or compositions on disease development in an embryo or larvae system, comprising the steps of:

providing a plurality of start biosystems, said start biosystems being selected from living eggs or embryos of aquatic developing chordates, and said start biosystems being in the stage prior to 22 hours post fertilization;

introducing one or more replicating entities capable of effecting disease development in the yolk of at least a set of said start biosystems;

exposing said set of said start biosystems to said functional chemical compounds or compositions;

exposing at least a subset of said start biosystems to one or more gene-function-modifying molecules;

allowing said start biosystems to develop to result in a plurality of embryos or larvae;

determining a response in said embryos or larvae;

correlating said gene-function-modifying molecules and said response; and

identifying gene-function-modifying molecules counteracting the effect of said functional chemical compounds or compositions on disease development.

The term “functional chemical compounds or compositions” as used herein refers to chemical compounds or compositions that have demonstrated an effect on disease development effected by introduction of one or more replicating entities into the yolk of the start biosystems. Said one or more replicating entities preferably include at least one pathogen. A functional chemical compound or composition may also be referred to as a “pharmaceutical drug candidate”.

The method may be for determining a target of one or more functional chemical compounds or compositions. The resultant of this method is the knowledge which gene-function-modifying molecule counteracts the effect of the functional chemical compound or composition on disease progression, i.e., what is the target of the functional chemical compound or composition. In a preferred embodiment, when searching for pharmaceutical drug candidates, said functional chemical compounds or compositions inhibit, slow down or halt disease development effected by the introduction of said one or more replicating entities. An effective gene-function-modifying molecule may counteract the effect of the functional chemical compound or composition, and may stimulate, speed up or start disease development again in the presence of the functional chemical compound or composition. In order to be able to elucidate the mechanism-of-action of the functional chemical compound or composition, knowledge of the gene-function-modifying molecule, and in particular its target, may be important.

A “gene-function-modifying molecule” as used herein refers to a molecule modifying or eliminating the function of a gene. It includes, without limitation, gene-silencing molecules, such as siRNA and sense- and antisense-DNA. Preferably, it is known which gene is affected by such gene-function-modifying molecule. In a suitable embodiment, said gene-function-modifying molecules are gene-silencing molecules.

In case of high-throughput screening of gene-function-modifying molecules, in an embodiment a single type of replicating entities and a single functional chemical compound or composition is added to most if not all of the start biosystems, e.g. in a 96-well format. The start biosystems of each of said 96 wells may be exposed to a different gene-function-modifying molecule. However, in order to allow statistical analysis at least about 10 to about 15 start biosystems are preferably exposed to a single gene-function-modifying molecule.

The gene-function-modifying molecule may be added externally to the start biosystems; however, it is preferred that said gene-function-modifying molecule is introduced into said start biosystems.

In an embodiment, said one or more replicating entities, said functional chemical compounds or compositions and said one or more gene-function-modifying molecules are introduced simultaneously. This embodiment allows 3-component injection which is highly time and cost efficient.

In an embodiment of the methods of the invention, said plurality of start biosystems are provided via a flow through system.

In another embodiment of the methods of the invention, said plurality of start biosystems are provided via a holding system is which said plurality of start biosystems are retained at substantially fixed positions.

In an embodiment, said replicating entities are introduced in at least about 300 start biosystems per hour, in an embodiment in at least about 1500 start biosystems per hour.

In an embodiment of the methods of the invention, the methods are for high throughput screening of said chemical compounds or compositions, wherein:

a) said providing comprises positioning an array of a plurality of said start biosystems in a holder in which said embryos are retained at their position;
b) said introducing comprises injecting the yolk of said plurality of said start biosystems in said holder with said one or more replicating entities.

In an embodiment, said replicating entities are introduced in the presence of carrier compounds.

In an embodiment, the replicating entities are embedded in carrier material, in an embodiment embedded in inert non-immunogenic fluid polymers such as PVP, in an embodiment embedded in inert non-immunogenic solid polymers such as cellulose sulphate, chitin, chitosan or plastic, in an embodiment embedded in inert non-immunogenic solid photo-degradable polymers such as plastics, in an embodiment embedded in a hydrogel.

In an embodiment, said response is measurable at the physical level, transcriptome level, proteome level and metabolome level, e.g. at the optical level. Preferably, said response is measurable within five days after introducing said one or more replicating entities.

### System for High Throughput Screening of Chemical Compounds or Compositions

In a further aspect, the present invention provides a high throughput screening system for a set of chemical compounds or compositions using a plurality of start biosystems having a yolk, said start biosystems selected from the group consisting of living eggs and living embryos of aquatic developing chordates, said system comprising:

a controller;

a transporter, operationally coupled to said controller, for passing start biosystems individually past an introduction position;

an injector, operationally coupled to said controller, adapted for intrayolk introduction of at least one living entity in at least a set of said start biosystems at said introduction position;

an exposure system for exposing at least a set of said start biosystems to one or more of said chemical compounds or compositions, said exposure system operationally coupled to said controller;

a first detector, operationally coupled to said controller, for measuring a first response of said each of said start biosystems and transmitting the measurements to said controller, said controller storing said measurements coupled to the replicating entity introduced into a biosystem and the chemical compound or composition that biosystem was exposed to.

In an embodiment, said transporter comprises a holder comprising at least one cavity, dimensioned for holding one of said start biosystems in a substantially fixed position.

In an embodiment, said transporter is adapted for passing at least 300 start biosystems per hour past said introduction position, in an embodiment at least 1500 start biosystems per hour.

In an embodiment, said transporter comprises an actuator for displacing said holder for passing start biosystems individually past said introduction position.

In an embodiment, said system further comprises a second detector, operationally coupled to said controller, for identifying a second property of each of said start biosystems and storing said second property with an identifier of said start biosystem in a memory of said controller.

In an embodiment, the system further comprises a biological safety cabinet confining said transporter and said injector, in an embodiment said safety cabinet complying at least to the biosafety level 2 requirements (BSL-2), in particular to the biosafety level 3 (BSL-3) requirements. In such case, the system may be integrated into a single set-up allowing operation by a remote control. This allows testing of highly pathogenic organisms or viruses that have to be contained in specially shielded environments to prevent escape of the pathogens.

In an embodiment, said transporter comprises a holder comprising a plurality of cavities at a regular spacing, each cavity having a size adapted for holding one starting biosystems at a substantially fixed position.

In an embodiment, said holder comprises a cover slide with injection through holes at the positions of said cavities for preventing said start biosystems from leaving said cavities and allowing said injector to deliver a replicating entity in said yolk.

In an embodiment, said transporter comprises a groove in which start biosystems are situated side by side resulting in a regular spacing, in an embodiment said start biosystems are prevented from leaving said groove by a cover slide with a slit at the position of said groove, in a further embodiment said slit dimensioned for allowing said injector to deliver a replicating entity in said yolk.

In an embodiment, said transporter comprises a flow-through channel.

In an embodiment, said system comprises a rotating disc with cavities around its circumference, each cavity for holding a start biosystem.

In an embodiment, said transporter comprises at least one cavity for holding a start biosystem, said cavity coupled to a underpressure channel debouching in said cavity for in operation holding a start biosystem at a substantially fixed position in said cavity.

The injector may comprise one or more of the following components: an automated stage positioner, such as the Märzhäuser MT mot. 200×100−1 mm MR; a microplate holder that can be attached to the stage positioner; a controller for the automatic stage positioner that is accessible via an RS232 port, such as the Märzhäuser Tango2-desktop controller; a programmable micromanipulator that is accessible via an RS232 port, such as the Eppendorf InjectMan NI2; a programmable injector that is accessible via an RS232 port, such as the Eppendorf Femtojet Express; an external compressor that provides the air pressure for the injector; software running from a PC to control the coordinated movement of the stage positioner, the micromanipulator and the injector via the RS232 ports; a capillary holder for connecting the capillary to the micromanipulator; a glass or steel capillary that is attached to the capillary holder and the injector; and a system for measuring capillary intactness and pressure.

The holder may be a custom-made embryo holder. Non-limiting examples of such custom-made embryo holder include the following:—a 1536-wells microplate, e.g., made of stainless steel. Both the diameter and the depth of the wells may be about 1 mm and each well is preferably capable of containing a single zebrafish embryo at a time only. The bottom of the wells contains a cavity with a diameter of about 300 μm.

The 1536-well microplate may be further equipped with a custom-made injection lid. The lid contains 1536 holes with a diameter of about 300 μm that exactly cover the center of the 1 mm diameter holes holding the embryos. One of said a first detector and second detector may be a prescreening detector for filtering out embryos that were injected in a faulty manner compromising further development of the embryo. In a highly suitable embodiment, such prescreening detector may be a COPAS™ BioSorter. The BioSorter may e.g. be used for viability screening, screening for granuloma formation, immune cell screening, and validation of the technology with low throughput microscopy.

The invention is hereinafter described in more detail with reference to the drawings.

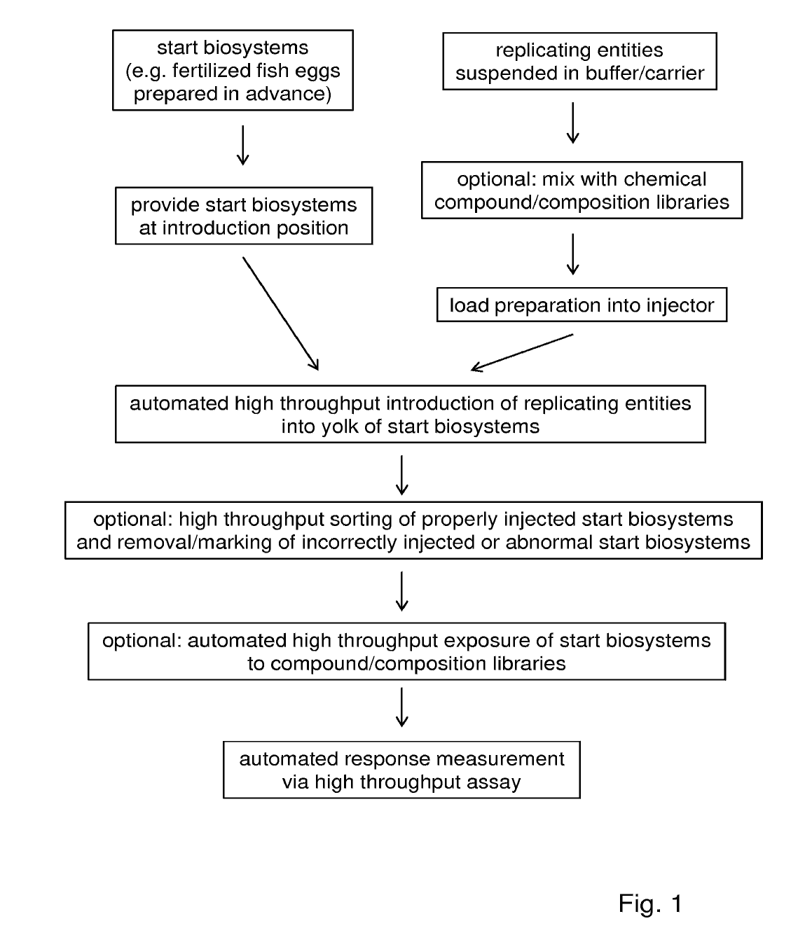

In FIG. 1 a flow chart is depicted which shows an example of the method of the invention. In this example, the start biosystems are fertilized fish eggs. These fertilized eggs are prepared in advance in this embodiment. Also, in a suspended phase replicating entities are here prepared in a buffer or in a carrier to avoid damage during injecting. In an embodiment, the chemical compound or composition may be added to the replicating entities. This preparation is loaded into an injector in the next step. Furthermore, the start biosystems are provided at the introduction position. In the next step, the start biosystems and replicating entities come together. In a next step of this embodiment, the start biosystems having the replicating entities introduced in the yolk are sorted and incorrectly injected start biosystems may be removed or may be indicated as incorrectly injected or abnormal start biosystems. In the next step of this embodiment, the start biosystems may be exposed to chemical compound or composition libraries. In some embodiments, the exposure can be combined with the introduction of the replicating entities in the yolk. Next, a response is determined. In this embodiment, the measurements are preformed in a high thoughput assay.

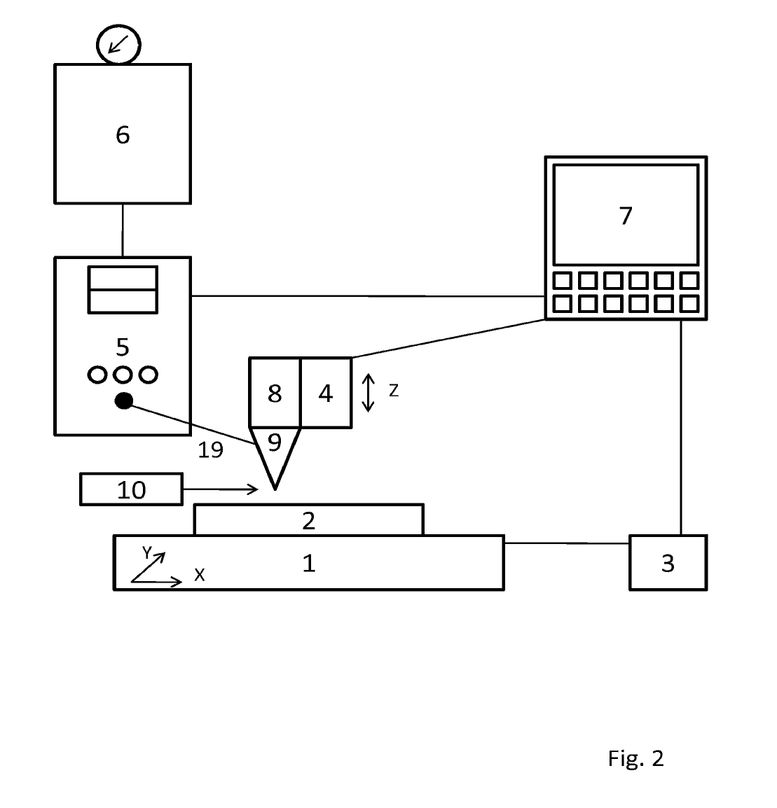

In FIG. 2, an example of a high throughput system is schematically shown. The system comprises the following components are used in this embodiment. The transporter comprises an automated stage positioner 1, such as the Märzhäuser MT mot. 200×100-1 mm MR, that controls the horizontal movements (x-y) of the starting biosystems attached to or confined in a microplate holder 2 which also is part of the transporter. The microplate holder 2 is in this embodiment attached to the stage positioner 1 and serves to connect the embryo holder to the stage positioner 1.

The system further comprises a controller. In this embodiment, the controller comprises in this embodiment a general purpose computer 7. In this embodiment, this general purpose computer 7 controls a controller (3) for controlling the automatic stage positioner 1. The controller 3 for controlling the stage positioner is for instance a Märzhäuser Tango2-desktop controller that is driven by software running on the general purpose computer 7 via an RS232 port. It serves to control the horizontal movements of the stage positioner 1.

The system in this embodiment further comprises an injector for the intra yolk introduction of the living entity in the start biosystems. In this embodiment, the injector comprises a programmable micromanipulator 4, such as the Eppendorf InjectMan NI2, that is also controlled by software running on the general purpose computer 7. via an RS232 port and serves to control the vertical movements (z) of a capillary 9. In this embodiment, the injector further comprises a programmable injector 5, such as the Eppendorf Femtojet Express, that is controlled by software running on PC 7 via its RS232 port and serves to provide a specific volume of fluid to the capillary 9 in order to introduce it into the yolk of the start biosystems. The programmable injector 5 is driven by an external compressor 6 that provides the air pressure for the programmable injector 5.

In order to coordinate the transporter providing the start biosystems at the introduction position, Software running on the general purpose computer 7 controls the movements of the stage positioner/controller 1, 3 and of the programmable micromanipulator 4 and the programmable injector 5, here all via the RS232 ports of the general purpose computer 7. The injector further comprises in this embodiment a capillary holder 8 that serves to connect the capillary 9 to the programmable micromanipulator 4. The capillary can be a glass or steel capillary 9 that is attached to the capillary holder 8 and the programmable injector 5.

The injector further comprises method measuring system for measuring capillary intactness its pressure 10. The injector further comprises tubing 19 that serves to connect the capillary 9 to the programmable injector 5.

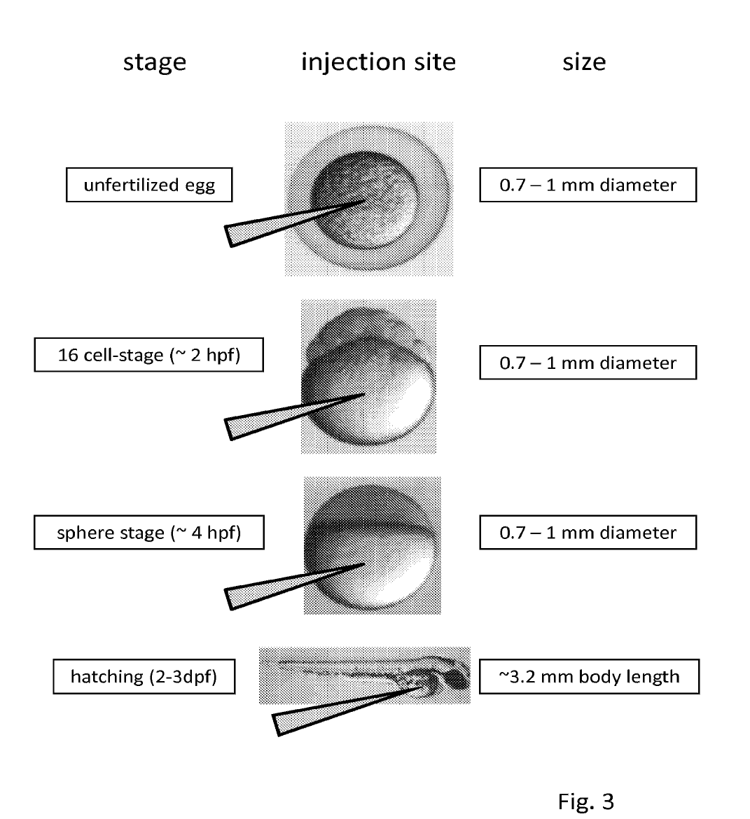

The start biosystems, for instance fish eggs, are fertilized according to standard protocols, e.g. using breeding tanks with dividers or in vitro fertilization techniques. At various stages after fertilization, which are outlined in FIG. 3, the start biosystems are transferred to a custom-made embryo holding device. The embryo holding device serves to hold the embryo in a fixed position at the introduction position during the introduction of the replicating entities in the yolk of the start biosystems. The pictures of the various stages further illustrates that at these stages, especially at the first stages, the yolk is larger than the rest of the embryo.

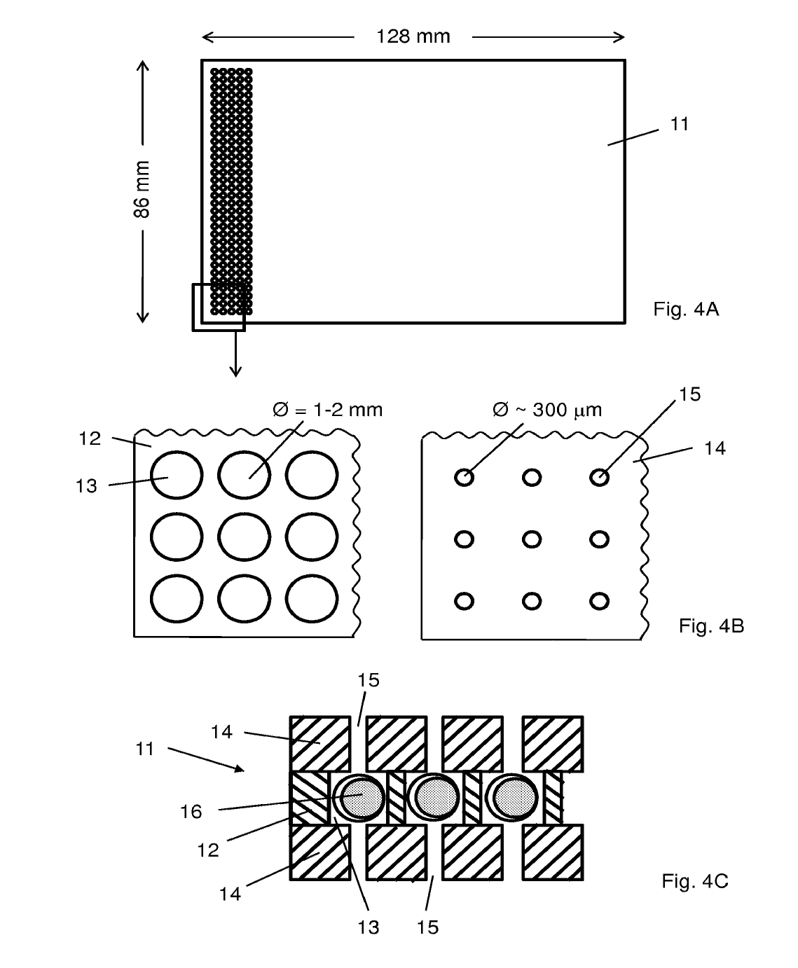

In one embodiment, depicted in FIGS. 4A-4C, the start biosystems are mounted at high density on or in a carrier device 11 at regular spacing. Using cover slides 14 keep these start biosystems in the device during the injection. In the case of sticky eggs, e.g. carp eggs (as detailed later), in an embodiment embryos can be held in place via their own capacity to stick to other materials. The carrier devices are made of metal, plastic, ceramic or glass. The carrier device with embryos is placed into the microplate holder of the stage positioner (FIG. 2). In one aspect of the invention as shown in FIGS. 4A-4C, the carrier device 11 has a microplate format (standard outer dimensions: 128 mm×86 mm) comprises a central plate 12 with more than thousand regularly spaced holes 13, each of which can hold one embryo 16. Both the diameter and the depth of the wells or holes 13 (e.g. 1-2 mm) are dependent on the start biosystems and each well can contain only one start biosystem, for instance a fish embryo, at a time. The start biosystems 16 are prevented from leaving the holes 13 by a bottom slide 14 and a cover slide 14. These cover slide 14 and bottom slide 14 contains holes 15 that are smaller than the holes 13 in the central plate 12. The diameter of these smaller holes 15 is for instance between about 200-400 microns. In an embodiment, these holes 15 have a diameter of about 250-350 microns, for instance 300 micron. These holes 15 are positioned to cover the center of the holes 13 in the holding slide 12 or central plate 12. This permits entry of an injection needle into the yolk of a start biosystem 16. In one aspect of the embodiment, the bottom slide 14 contains holes 15 with a diameter of about 200-400 microns, in an embodiment about 250-350 microns, for instance about 300 microns. These holes are used for underpressure-assisted fixation of the start biosystems during assembly of the slide sandwich. It may also be used during the introduction of the replicating entities in the yolk.

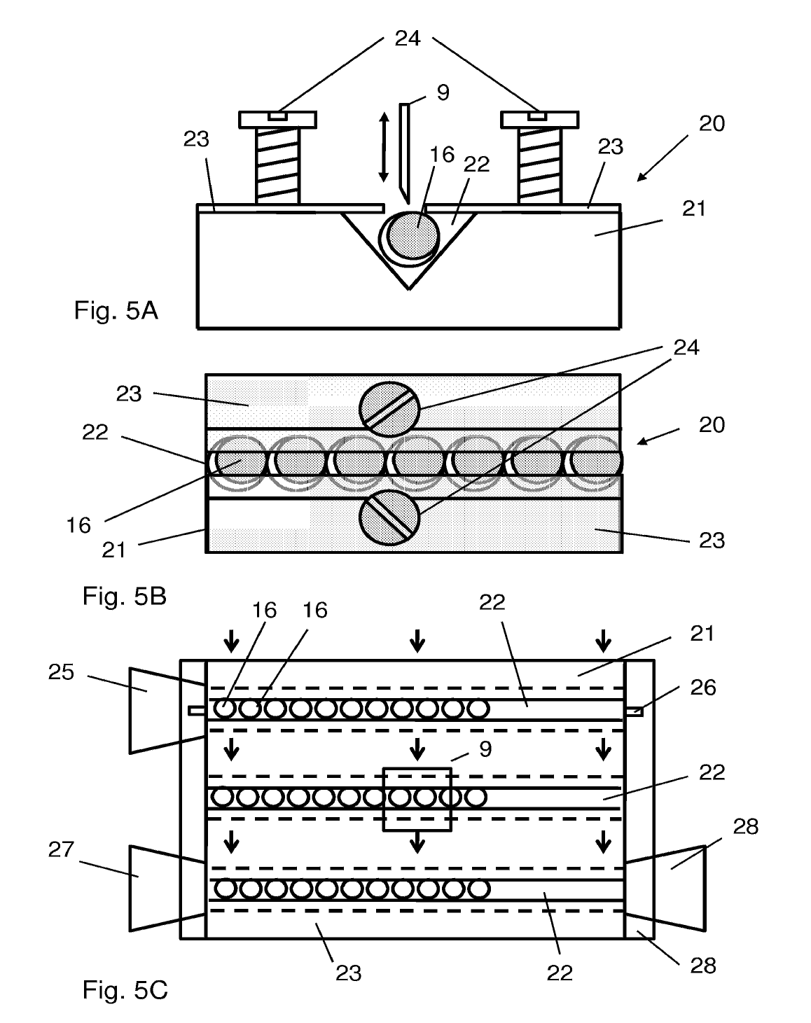

In another embodiment of the transporter, the holding device comprises a half open channel in which embryos are aligned in a row. In this row, they can for instance be accessed by a needle, but does not allow the embryos to get outside of the channel. In an embodiment, this is shown in FIGS. 5A-5C. Start biosystems are loaded into the holding device via a funnel-shaped fill point using a standard pipette tip. The width of the injection slit can be adjusted with cover slides using fixing screws. The holding device can be placed for instance on the microplate holder of the stage positioner of FIG. 2. The holding device 20 of the transporter comprises a plate 21 in this embodiment provided with a V-shaped groove 22. In such a groove, the start biosystems 16 are limited in their sideward movements. The plate 21 in this embodiment is covered with a cover slide 23. In this embodiment, the space between cover slides 23 is set by positioning screws 24.

In FIG. 5C, the holding device has several grooves 22 transverse to the transport direction, indicated with the arrows. In the center, an injection position is indicated. The top row is at the left side coupled to a filling adapter 25. The rows shift in the drawing in downward direction. Each time, a next row is positioned at the injection position. Next, the injector 9 moves from left to right or vice versa to inject all the start biosystems in a row. After injecting a row, the row is shifted and via exit adapter 28 the injected start biosystems leave the groove 22.

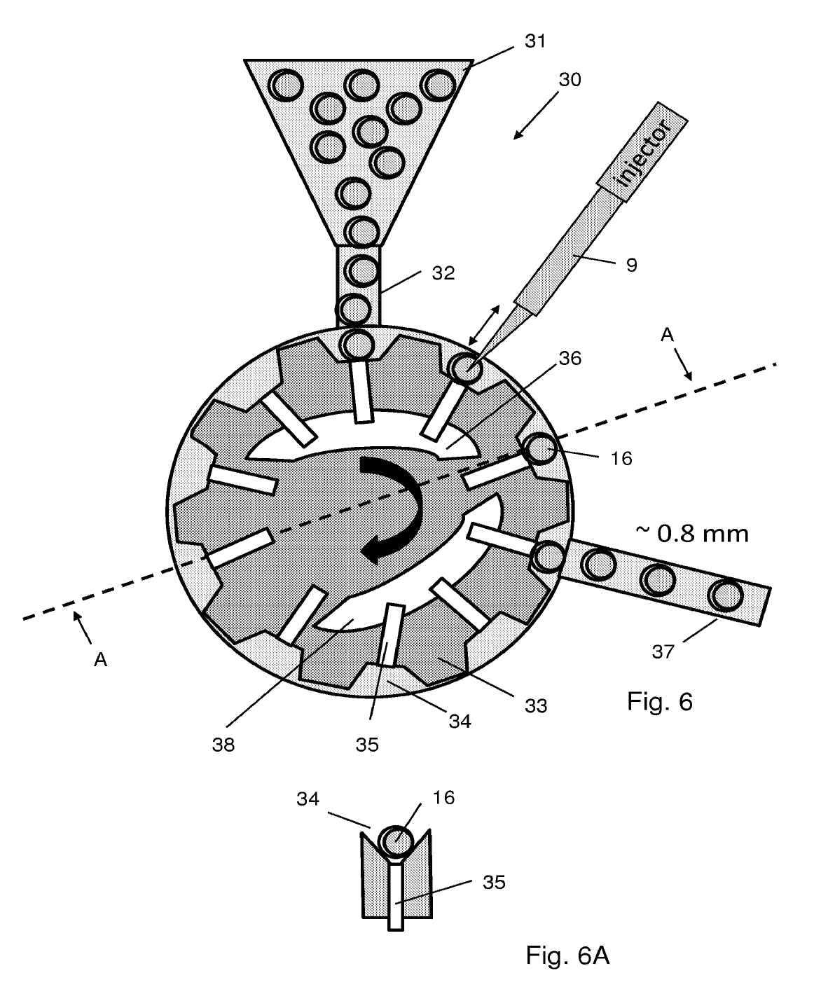

In another embodiment of the system, in particular the transporter, depicted in FIGS. 6 and 6A, the holding device 30 comprises a rotating carousel 33 which allows start biosystems 16 to be fixed in a high throughput manner and subsequently injected. The start biosystems 16 enter the rotating carousel 33 via a funnel-shaped fill point 31 coupled to a capillary 32. The rotating carousel 33 comprises a disk 33, formed as a cogwheel. Each compartment 34 of the cogwheel 33 can hold one start biosystem 16 that is fixed to in its compartment or cavity 34 by underpressure, provided via a channel 35. Channels 35 each couple a compartment 34 to a central cavity 36, 38. As the rotating carousel 33 rotates, the channels are coupled to an underpressure coupling at the position of the filling station 31, 32 and the injector 9 in order to hold the starting biosystems 16 in their compartment 34 at a fixed, defined position. As the rotating carousel 33 continues, the central cavity couples to a overpressure. This overpressure removes the injected start biosystems 16 from their compartment 34 and brings them in an outlet capillary 37. In an embodiment, the cross section of the outlet capillary is about 0.7-1 mm in cross section, in particular about 0.8 mm. Thus, after intrayolk injection, the start biosystem 16 is released from the cogwheel by pressure.

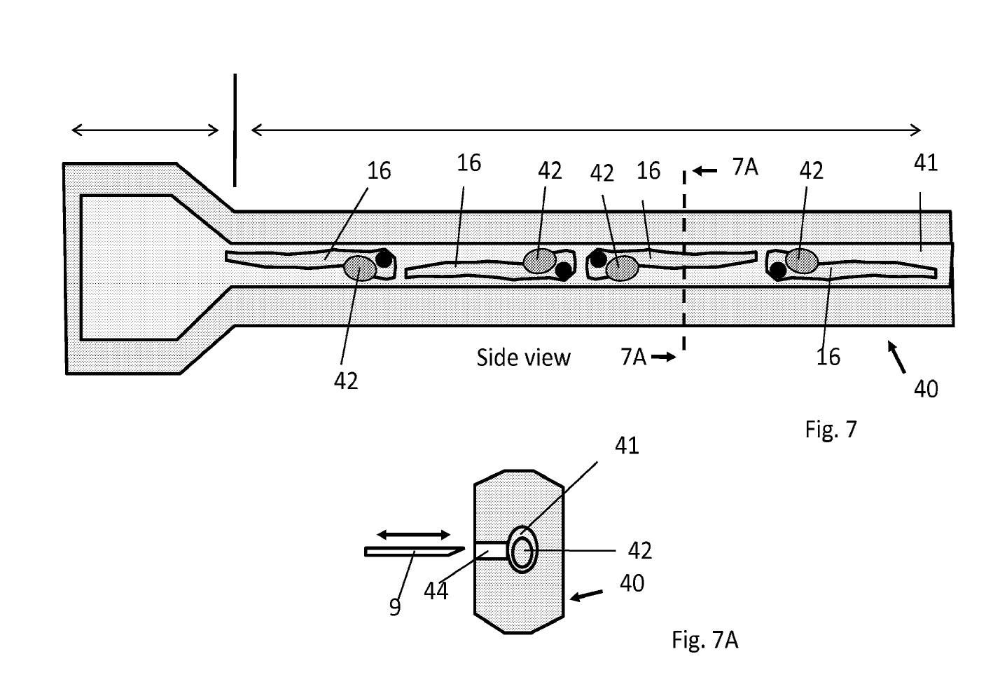

In yet another embodiment of the transporter, shown in FIGS. 7 and 7A the holding device 40 comprises a flow-through capillary or channel 41 designed in such a way that only one start biosystem 16 can pass at one time. It is subsequently injected into the yolk 42 at an introduction position via an injection hole 44 located substantially perpendicularly to the end of the capillary. FIG. 7A shows the flow through channel 41 in cross section. In this embodiment, the cross section of channel 41 is non-round. In particular, the channel is elliptic in cross section. This further improves the fixed orientation of the start biosystems 16.

Identification of Marker Genes, Marker Proteins and/or Marker Metabolites

In a further aspect, the present invention relates to a method for identifying marker genes, marker proteins or marker metabolites characteristic for a specific disease or situation, said method comprising the steps of:

providing a plurality of start biosystems, said start biosystems being selected from living eggs or embryos of aquatic developing chordates, and said start biosystems being in the stage prior to 22 hours post fertilization;

introducing one or more replicating entities capable of effecting said specific disease or situation into the yolk of at least a set of said start biosystems;

determining a transcriptome, proteome or metabolome in at least said set of start biosystems;

comparing the transcriptome, proteome or metabolome of biosystems in which replicating entities have been introduced with the transcriptome, proteome, or metabolome in biosystems in which no replicating entitities have been introduced; and

identifying marker genes, marker proteins or marker metabolites for said specific disease or situation.

The method of the invention may be applied for any replicating entity which is pathogenic, resulting in disease marker genes. Alternatively, the method may be applied for identifying probiotic marker genes.

The skilled person is well aware of methods to identify marker genes for a specific disease or situation.

Useful embodiments of this method are set forth above in relation to the method for screening chemical compounds or compositions, and apply mutatis mutandis to this method.

The terms “transcriptome”, “proteome”, and “metabolome” are well known in the art. As herein used, these terms have their usual meaning

The transcriptome is the set of all messenger RNA (mRNA) molecules, or “transcripts,” produced in one or a population of cells. The term can be applied to the total set of transcripts in a given organism, or to the specific subset of transcripts present in a particular cell type. Unlike the genome, which is roughly fixed for a given organism, the transcriptome can vary with external environmental conditions. Because it includes all mRNA transcripts in the cell, the transcriptome reflects the genes that are being actively expressed at any given time, with the exception of mRNA degradation phenomena such as transcriptional attenuation.

The proteome is the entire complement of proteins expressed by a genome, cell, tissue or organism. More specifically, it is the set of expressed proteins at a given time under defined conditions. The term has been applied to several different types of biological systems. A cellular proteome is the collection of proteins found in a particular cell type under a particular set of environmental conditions such as exposure to hormone stimulation. It can also be useful to consider an organism's complete proteome, which can be conceptualized as the complete set of proteins from all of the various cellular proteomes. This is very roughly the protein equivalent of the genome. The term “proteome” has also been used to refer to the collection of proteins in certain sub-cellular biological systems. For example, all of the proteins in a virus can be called a viral proteome. The proteome is larger than the genome, especially in eukaryotes, in the sense that there are more proteins than genes. This is due to alternative splicing of genes and post-translational modifications like glycosylation or phosphorylation. Moreover the proteome has at least two levels of complexity lacking in the genome. When the genome is defined by the sequence of nucleotides, the proteome cannot be limited to the sum of the sequences of the proteins present. Knowledge of the proteome requires knowledge of (1) the structure of the proteins in the proteome and (2) the functional interaction between the proteins.

Metabolomics is the systematic study of the unique chemical fingerprints that specific cellular processes leave behind—specifically, the study of their small-molecule metabolite profiles. The metabolome represents the collection of all metabolites in a biological organism, which are the end products of its gene expression. Thus, while mRNA gene expression data and proteomic analyses do not tell the whole story of what might be happening in a cell, metabolic profiling can give an instantaneous snapshot of the physiology of that cell.

As used herein, marker genes, marker proteins or marker metabolites characteristic for a specific disease or situation are differentially expressed in biosystems in which replicating entities have been introduced in comparison to the levels of the same genes, proteins or metabolites in biosystems in which no replicating entities have been introduced. For example, marker genes may be present in the transcriptome of biosystems in which replicating entities have been introduced, whereas they are not present in the transcriptome of biosystems in which no replicating entities have been introduced. Alternatively, marker genes may be expressed in the transcriptome of both biosystems in which replicating entities have been introduced and biosystems in which no replicating entities have been introduced, but may be markedly upregulated or downregulated in the transcriptome of biosystems in which replicating entities have been introduced. Similarly, marker proteins may be present in the proteome of biosystems in which replicating entities have been introduced, whereas they are not present in the proteome of biosystems in which no replicating entities have been introduced. Alternatively, marker proteins may be present in the proteome of both biosystems in which replicating entities have been introduced and biosystems in which no replicating entities have been introduced, but may be markedly upregulated or downregulated in the proteome of biosystems in which replicating entities have been introduced. The same holds true for marker metabolites.

The marker genes, marker proteins, or marker metabolites are characteristic for a specific disease or situation. The marker genes, marker proteins, or marker metabolites may e.g. be specific for injection of Mycobacteria, probiotic lactobacilli, Trypanosomes, cancer cells, yeasts, fungi, Gram-negative bacteria, viruses, and the like.

The method comprises a step of providing a plurality of start biosystems, said start biosystems selected from living eggs or embryos of aquatic developing chordates, as explained above.

In a further step, one or more replicating entities capable of effecting said specific disease or situation are introduced into the yolk of at least a set of said start biosystems.

Particularly in case of a high throughput system, the start biosystems may be divided into sets of start biosystems, in which replicating entities may or may not be introduced. A high throughput system would allow simultaneous recording of both “challenged” biosystems and “unchallenged” biosystems by dividing the plurality of starts biosystems into sets of start biosystems in which replicating entities are to be introduced (“challenged”) and start biosystems in which no replicating entities are to be introduced (“unchallenged”). In this case, the transcriptome, proteome or metabolome of both challenged and unchallenged start biosystems may be compared in the same experiment using the same chemicals.

In an embodiment, unchallenged start biosystems receive the same treatment as challenged start biosystems, with the exception of the introduction of replicating entities. Replicating entities are often introduced in an aqueous dispersion comprising buffer and optionally carrier material. The introduction procedure itself, whether it be injection or ballistic procedures or any other method known in the art, and other components but the replicating entities comprised in the aqueous dispersion that is introduced into the challenged biosystems may have an effect on the transcriptome, proteome, or metabolome. This effect is not due to the replicating entities, but is an accessory effect, and cannot be attributed to introduction thereof into the challenged start biosystem. To correct for such accessory effects, the transcriptome, proteome, or metabolome of challenged start biosystems are preferably compared to the transcriptome, proteome, or metabolome of unchallenged start biosystems which have received the same introduction procedure as the challenged start biosystems, albeit without the replicating entities. In this case, any differences between the transcriptomes, proteomes or metabolomes can be ascribed primarily to introduction of the replicating entities.

In order to identify statistically significant differences in the transcriptome, proteome or metabolome, it is preferred that the replicating entities are introduced in an amount of start biosystems allowing statistical analysis. Similarly, it is preferred that the method of the invention is carried out with a sufficient amount of start biosystems in which no replicating entities are introduced to allow statistical analysis. The differences in the transcriptome, proteome and metabolome would then be considered statistically relevant.

From total genome based data sets subsets of markers can be defined that, because of their lower complexity, are easier to apply in high throughput screening. These markers are either read out by DNA-based assays, such as PCR and restriction enzyme analysis, RNA-based assays, such as RT-PCR, RT-MLPA, or promoter-based transgenic fluorescent or luminescent reporter constructs, antibodies (e.g. ELISA), and sensors for particular metabolic compounds.

### Disease and Probiotic Marker Sets

Marker genes were selected that can be used to analyze the genetic response to a particular treatment in zebrafish and other fish species. These genes were compared to whole transcriptome sequence data sets of zebrafish and carp embryos that were introduced in the yolk. These transcriptome sets were compared with proteome data. Of each category a set was chosen that can be most optimally useful for high throughput purposes on basis of the following criteria: reproducibility, fold change difference after treatment, and applicability in all fish species. The sets are summarized in table 1, which shows a selection of marker genes demonstrating specific expression changes upon introduction of either BCG (Bacille Calmette Guerin (BCG) vaccine for tuberculosis, which containes a live attenuated (weakened) strain of Mycobacterium bovis), Rhizobium, Lactobacillus casei shirota (“Yakult”), Trypanosomes, Mycobacterium leprae, Mycobacterium smegmatis, and Mycobacterium marinum. The set comprising 94 marker genes is sufficient to determine which of one or more replicating entities selected from the group consisting of BCG, Rhizobium, Lactobacillus casei shirota (“Yakult”), Trypanosomes, Mycobacterium leprae, Mycobacterium smegmatis, and Mycobacterium marinum were introduced in the start biosystems.

Thus, the method also relates to a method for determining the presence or absence of BCG, Rhizobium, Lactobacillus casei shirota (“Yakult”), Trypanosomes, Mycobacterium leprae, Mycobacterium smegmatis, and/or Mycobacterium marinum in a sample, said method comprising the step of: a) providing a plurality of start biosystems, said start biosystems being selected from living eggs or embryos of aquatic developing chordates; b) introducing said sample in the yolk of at least a set of said start biosystems; c) allowing said start biosystems to develop to result in a plurality of embryos or larvae; d) determining the expression of marker genes as depicted in Table 1 in said embryos or larvae, and e) correlating the expression of said marker genes to the presence or absence of BCG, Rhizobium, Lactobacillus casei shirota (“Yakult”), Trypanosomes, Mycobacterium leprae, Mycobacterium smegmatis, and/or Mycobacterium marinum. The same set of marker genes can also be used to type the presence of other replicating entities in the yolk of said biosystems.

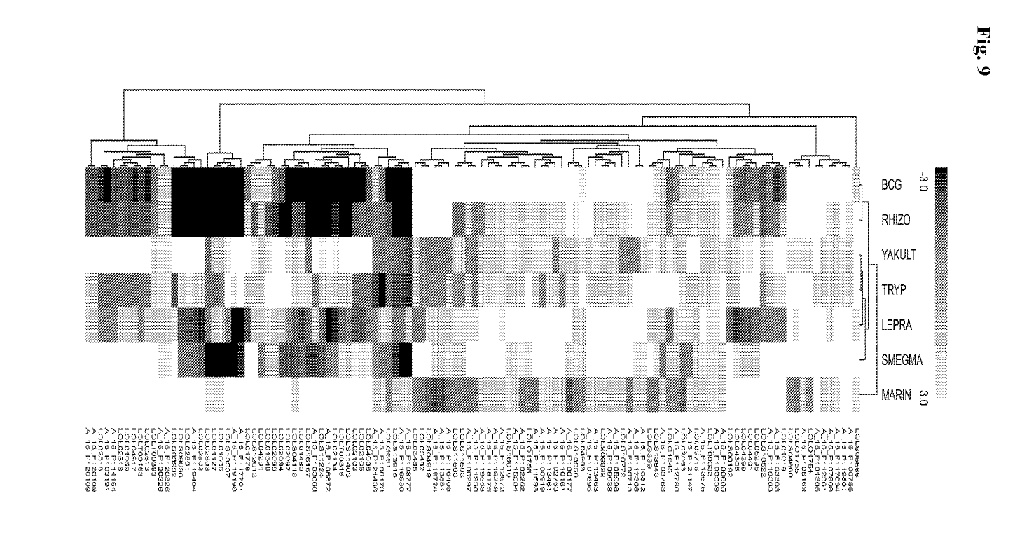

The marker genes were primarily identified by micro-array screens with zebrafish embryos but data was also confirmed with yolk injection in carp fish and RNA deep sequencing of carp embryos injected with replicating entities. The set has been selected based on methods and criteria described in the text and the legend of FIG. 9. The combination of these probes, as a fingerprint, will determine the specificity of the response. A minimized set of representatives was made in order to make high throughput applications quicker and cheaper. Furthermore, the use of a smaller subset of marker genes will facilitate bioinformatics analyses compared to large gene sets. A combination of the marker genes can be either used in RT-PCR analyses, RT-MLPA sets or micro-array based assays, but other techniques are also possible. The proteins encoded by the transcripts can be used in antibody assays or proteomic read out methods. The category of genes which are mentioned as category “predicted immune genes” is tentative since there is not yet evidence of the function of these genes in the immune system in fish since no knock-out or knock down studies of these genes have been performed in fish species yet. Furthermore, the expression of these genes has not been related to be specific markers for a particular disease. Thus, the present inventors have found for the first time that using probes based on the nucleotide sequences of these genes is useful for providing a diagnosis for the kind of living entity that has been injected into fish, in particular in a yolk injection system. For some of the living entities that are injected it is not needed to use the entire set of probes. The ds-red probe responds to the RNA injected living entities RNA in the case that these were genetically modified with a gene construct containing the ds-red gene or, alternatively the m-cherry gene that was also used as a marker in our studies. Of one of the probes there is no translational product identifiable since it is an antisense probe for CCL24 chemokine. This shows that antisense probes are also highly useful for identification of the living entity after injection in the yolk. We also have identified miRNAs that are up-regulated after yolk injection of replicating entities (Table 2), for instance, miRNA 146a (dre-miR-146a). These may therefore also be highly useful markers either in micro-array analysis, deep sequencing of RNA and RT-MLPA.

TABLE 1
A selection of marker genes demonstrating specific expression changes upon introduction
of either BCG (Bacille Calmette Guerin (BCG) vaccine for tuberculosis, which containes
a live attenuated (weakened) strain of Mycobacterium bovis), Rhizobium, Lactobacillus
casei shirota (“Yakult”), Trypanosomes, Mycobacterium leprae, Mycobacterium smegmatis,
and Mycobacterium marinum.

| | Accession | Manual | |
|---|---|---|---|
| Category | original | annotation | Sequence |
| 1:Predicted immune system | NM_200637 | adam8a | CAAGTTTGCAATGATCTCAGCTGGGCTGATTTTA |
| | | | CTCTTTAAATGTGAGAATGCTCTCTT |
| 1:Predicted immune system | XM_688922 | bcl3 | AGCAGTGACCAATCAGACATATCTACTGTGAGTG |
| | | | TCAACAGTGAAGAAAGAGGTGTGAGT |
| 1:Predicted immune system | BC076048 | cd36 | CGGCCCATCCGACGATATTGCACTTTTGAACAAA |
| | | | ATCAAGGAGCACACAATTATACCTAT |
| 1:Predicted immune system | AY340959 | il1b | GACCATTAAAGCTGGAGATCCAAACGGATACGAC |
| | | | CAGCTGCTGTTCTTCAGGAAGGAGAC |
| 1:Predicted immune system | NP_001018628 | il22 | TATGAAATACCCAATGATTCGCAATGTGAGGGAG |
| | | | GGTCTGCACAGAGTCGAGCAAGAATT |
| 1:Predicted immune system | NP_001018635 | interleukin 26 | AGTGTTTTGCTGTGGATCAGTTCAGGCATGGACA |
| | | | GAAGAAAAACATACAAGAAGATTCAC |
| 1:Predicted immune system | CN507361 | Interleukin-8 | TGTCTGGACCCCTCTGCTCCATGGGTTAAGAAGA |
| | | | TCATTGATAGGATCATTGTCAAGTAA |
| 1:Predicted immune system | CK026195 | irak3 | TCATGAGCACGTTGACAAGCCTCTATCTTGGCAA |
| | | | GAACGGCTGAATATTATCAAAGGCAC |
| 1:Predicted immune system | NM_001048055.1 | lect21 | ACCGACTGCCACCATCAAACTTTGCCAACTTCTT |
| | | | TTGTGCCATTTATCAGATTAATTTCT |
| 1:Predicted immune system | NM_213123 | mmp9 | TCATAGGCACATGAGACGGGATGTTAGGCATATT |
| | | | TGTCCGTCAGCTTTACCATGGTCTTA |
| 1:Predicted immune system | AY324390 | nos2a | CATTCTTCTATTACCAGACTGATCCATGGCTAAC |
| | | | ACACAAGTGGAAAGATGAGAAGAAAG |
| 1:Predicted immune system | TC288443 | Plac8 | AACAGTTCTGAGAAAACTTTTTTCAAAGATTCAA |
| | | | AAGCAGTGAACAGAGTTGGTCTGCTT |
| 1:Predicted immune system | NM_001007167 | mhc2dab | TGGTACCAAACTGACAGCAGTGAGTCTAATGTTA |
| | | | CCTGATGACTACTGAAAGAAGACCTG |
| 1:Predicted immune system | AY427649 | tnfa | GCACTTCTACCCATGGTTGAAAATGATAACGGAA |
| | | | AGACCTTCTTTGGGGTGTTTGGTTTG |
| 1:Predicted immune system | AB183467 | tnfb | GACTAAGGCTAAGAGGCCTCCCTGCATCGTGATG |
| | | | ACTTGTTTTATATGTAAAACAAAGCC |
| 2:Predicted function | CN326771 | CCL24 | TTGACTTCCCAATTCCAGCCAACAAAATTATGTT |
| | | | TGTTGCGAGGACGTCTTCACGTTGCG |
| 2:Predicted function | CN326771 | CCL24 antisense | ACAGCAAGGTCAGCAGGAACTAGCTGAATTGAAC |
| | | | ACAAGGTGAACACCAAAAACAGCAGA |
| 2:Predicted function | CN170399 | CXCL11a | TGAAGATGTCTGTCTGTTGGCAATGAAAAGAGAG |
| | | | CACAGGAGGTCAAGAGTGGGAATTCT |
| 2:Predicted function | AF202722 | rgl2 | GAAGAGATACGAGGAGCTTTCTGACATCTTCTCA |
| | | | GAGAAAGACAACTATTCTCAGAGCCG |
| 2:Predicted function | NM_131397 | hsp70 | TCTTATTGCACAGTGTGTTGGTTCACTATCTACT |
| | | | TAAACATCTTGATACAGTAAAATGTT |
| 2:Predicted function | AW116618 | hspa41 | GCGTTTGAGGCATGTATGCGCTGTATGTTGATGT |
| | | | AGATCTGCAGTGTTTGATCGTGAGCG |
| 2:Predicted function | ENSDARP00000036582 | lectin | AATACTGGAGTGAAATGTTACAAGTTTTTCTCTC |
| | | | AGTCGGTTAGCTGGATCACAGCAGAG |
| 2:Predicted function | BG884044 | Haptoglobin | CATGTTTCGGCTCTACGCTCCAGGAGGATGGTGG |
| | | | GAGGATCACTGACTGCTTCTGTGCCC |
| 2:Predicted function | AL929435 | fos | GGTCTCTTCCACACCAAACACATCAATCACGACC |
| | | protein tyrosin | TCTTCCAGCAGCCTGCTGTTCTCCAG |
| 2:Predicted function | XM_692434 | phosphatase | GACACGATCTATGTCAACGCAATGGCTTTAAAAG |
| | | | ATTTTGAAAATTCAAGCCACACATGA |
| 2:Predicted function | NM_131163 | b2m | ATCACTGTACAGGGGAAAGTCTCCACTCCGAAAG |
| | | | TTCATGTGTACAGTCATTTTCCAGGA |
| 2:Predicted function | AW232464 | ctssb.2 | AGGAACGCAAGGATCGTGTAGATATGACCCATCC |
| | | | CAGCGTGCAGCAAACTGTACTTCTTA |
| 2:Predicted function | TC272380 | cyp2j28 | CTGAACAAATCCAGTAGATTTCATTCTCTCTTTA |
| | | | TTAGGGGACTTCTATTACAACAAACC |
| 2:Predicted function | NM_152960 | fabp10 | AAGAGCAAGAAGATCTGAAGCGTTTCACCATCAC |
| | | | TCTATTTAAATAAAGCTCTGACTGAC |
| 2:Predicted function | TC291162 | fads2 | ATGAAATTTAATTGGATTTCCTACTATTGGTCAT |
| | | | CGATTAAACGGATTAAACATCCCGGG |
| 2:Predicted function | BM103343 | hsp90a | AATCTCCTTTTTCTTGGCTCAAACAGATCGAATG |
| | | | GAGCTCGACGAGGAACAAAAAGCAGC |
| 2:Predicted function | NM_001020509 | ism2 | ATGTCTTTCCTTTTGAGATGGAAAATGGTACAGA |
| | | | ACCCTATGGCACAGATGTGGGCAGCT |
| 2:Predicted function | NM_213212 | myl9 | AGACAGCTAATAGACAGCAACAACAAGGCTAAGT |
| | | | TTGAACTCGCAGTGAAAATCTATTAT |
| 2:Predicted function | BI430378 | nos2 | CTGCGCAAACTCTCTACAGTGGCGTATCAGGAAG |
| | | | AGGATCGCAAACGACTTGAAGCGCTC |
| 2:Predicted function | NM_131175 | opn1lw1 | AAAGAGTGATTGGTAGATGCCTGCCCATGTACAG |
| | | | CATGTAATATGGTTCTATTTTTCTTG |
| 2:Predicted function | AI793569 | slc6a11 | ACACGAGGCTTATGTACAGTATGTCTTTGCATAG |
| | | | TTTAGGATGCATCAGTGTTTCTTATG |
| 2:Predicted function | CK705002 | sox21b | CACTGATATCCGGAAAGTCAGAGCTTTTACCTTT |
| | | | ACATCAAGGCATTATAATCATGATAC |
| 2:Predicted function | BG729009 | thbs1 | ATGAAGAACCGAACATCCTCACGTCAGTGTGCAA |
| | | | ACTGTTTATACAGATGGAATCGCCTC |
| 3:No known function | BG985584 | id:ibd5033 | GTCCACGCCGTAAACGATGTCTACTAGCTGTTCG |
| | | | TGTCCTTGTGACGTGAGTAGCGCAGC |
| 3:No known function | AJ299409 | id:ibd5150 | TCCCTGTCATATCGAACTCCAGACAGCCCTTGAC |
| | | | AAGATCACTAAATCACAGCAGAAACT |
| 3:No known function | CF999291 | LOC100002541 | CTGTGAGATCAAATGCAGTCATCCTGCTTCACAG |
| | | | TTACATTGATTTTACTACATTTTCTT |
| 3:No known function | BI533854 | LOC100002541 | ACCTCAATGGGCTATATGTGTGCTGCAAACCTGT |
| | | | GAGATCAAATGCAGTCATCCTGCTTC |
| 3:No known function | AW076838 | LOC100005016 | TCCACGAAACCTCTGTGAAATTCAGTGGCTCCAC |
| | | | AAATACTCACTTTCCACATCTTTAAG |
| 3:No known function | BG302802 | LOC100006917 | GCTCCCAGAAATGTGTAGATTTATCTGCATATTA |
| | | | TGAAAGCCTTGTGATAGGCTGAGAAC |
| 3:No known function | CK704956 | LOC553326 | TTTGACCACTTGTTGCTATATCATGTTGCACTTG |
| | | | GTTAGAGTACAGTTTTATGCTGAAAT |
| 3:No known function | BM860989 | LOC558967 | GTTACAGAACAACTCTAACTCTCTGAGTCAGAAG |
| | | | AAACTGGAGCTGGAGAACAGAGTCAC |
| 3:No known function | AL924126 | LOC561790 | GGTTTGTCGATATGGTCAACAGCATGTCAATAAA |
| | | | AACAAACCTAAAACCACTTCAAAAAA |
| 3:No known function | BC078367 | LOC562139 | GACCATCACTGCAAACTAAATCACCAAGCTAATG |
| | | | TTCATGGTCATAATGTTCATCAATAA |
| 3:No known function | BE200723 | LOC562155 | TTGGCTAATGATAGTTCAGAAATAAACCCCTAGC |
| | | | CGATCTCATGAACCGGTCACAAATGT |
| 3:No known function | CF996283 | LOC569924 | CATCTGCAACAGGGAATATAGGCCTGTATGTGGT |
| | | | ACAGATGGAATTACGTACCCAAACGA |
| 3:No known function | AI330682 | wu:fa91f08 | TCGTCTGCATCCTCGTGCCGTCAACTGCCTGAAG |
| | | | AAGAAGTGCGGACACACCAACAACCT |
| 3:No known function | AW281919 | wu:fb48d04 | TCTCTGTCCATCAGAGCCGGAGTGGTTTCAACTG |
| | | | TTGATCTCTTAGTGGTCTTATTGAGA |
| 3:No known function | AI522707 | wu:fb61a09 | CTCTGCTTGATGCTTCAACACTGCATAAATCATC |
| | | | TCCTCTGTGTGTGCTTTGTATGCGCT |
| 3:No known function | AW019476 | wu:fb63f09 | CTGAACATATGCTGCCTACTATTTCATGCTATAC |
| | | | CAGGGCTGATTCGAGACATTTGGAGG |
| 3:No known function | AI588213 | wu:fb97g06 | ATGACGATTCAGCACAACTCATCTCTTGAGGACT |
| | | | TTTATCCGTAGGCACACTTCTTTATG |
| 3:No known function | CN019915 | wu:fc49d01 | AACATCGAAATGCTTCACGTCTAAGTCTCGAAGC |
| | | | AGCCCTGCCTGGCCTTCTCGGTGGGT |
| 3:No known function | ENSDARP50439 | wu:fe15g08 | TAACCAGCAGGTACCAGCCTGGTTTGGTCCTGGT |
| | | | TCTGGACCCATATGGCTGGATGAGGT |
| 3:No known function | AW059054 | wu:fe16d09 | GTTCATCACCTGCTCGGCTCTCAAAAGATTATGT |
| | | | GACACTCTTTATCCAGACTGTGGAGC |
| 3:No known function | AW203046 | wu:fj08a04 | TGGACGTGGCCTGTTGGCTGATTCTTGTCTACCG |
| | | | CCGAATTAACAAATTGGTCATTCTAA |
| 3:No known function | ENSDART00000026822 | wu:fj08f03 | TCATAACCAAGTTTATGCAAAACTTGGTGACAAG |
| | | | GTAGATGGAGTGCTTTGTGATGGGCC |
| 3:No known function | AW279994 | wu:fj48a01 | TAGACACTGACATGCTCGGGAACAGTGGAGAAGC |
| | | | GGATCATGATTGTCTTGTGGGTCGAG |
| 3:No known function | AW281861 | wu:fj58c09 | AGATTTTGTTAGACCTGGTCCAGTCAAGTACTGC |
| | | | ATACAGAATATTACAAAGCCTTCTAG |
| 3:No known function | AW421098 | wu:fj92a04 | TTGCTCTTTTCCTCTCTTATTTTGTGCATGAGCA |
| | | | ACAACATTGATGAAATAATGTTTGCG |
| 3:No known function | NM_001005599 | zgc:103580 | CCTTTCTTTGACATATTTGCTTAATTCTTAATAT |
| | | | GTTATATTGAAGTTAAAGTGGTGTTA |
| 3:No known function | AW128435 | zgc:110285 | AGGCTTCAATTTGTCATCGGCACTACTTGATTCT |
| | | | TGCGTGAAACTTATAAATGTATATTG |
| 3:No known function | BI708803 | zgc:112052 | GTCTATACCTGAGAAATGACACAAACGCCGTTAA |
| | | | GCAGGGGTGAGTATTTGCGCTTGATT |
| 3:No known function | TC282253 | zgc:122979 | GTTAGCATACTTCTTTAACCATGACGATCTTGTA |
| | | | CTCGCGTACAGAACTGTCAGTGATTA |
| 3:No known function | AW280201 | zgc:136764 | CACGGGTTGAGAATGGTTCAATTCCCATGGTGTC |
| | | | GACATGCTAACGTAGAGTTGTCTACG |
| 3:No known function | TC294517 | zgc:152874 | GAGAACTCGTAATATTGTGATTTACTCGATGGTG |
| | | | TAATACTGCACTAGTGCTGTGCGAGT |
| 3:No known function | BI863963 | zgc:153009 | TTCTAGGCCGCTCAGTGGAAAAGTGACTCACACT |
| | | | TTCCTATTAATAAACAGTCCTTGCAG |
| 3:No known function | ENSDART00000020851 | zgc:153723 | GAAATGTTTCAGGATCTTCGTGGGTCTCTTCAGA |
| | | | AAGTGAGTCATTTCCTGCAGTGCACA |
| 3:No known function | BG305537 | zgc:154055 | TCCCCACCTAAATTTAAACCTATATTCTGTTCTC |
| | | | CGACAGATTGATTTGGTTCAATTATC |
| 3:No known function | BI877849 | zgc:172265 | ACAGTGCTTCAATTTTAGTGGGAACATTTAGTGG |
| | | | ACGTAAATTTCAGTACCGACTGGACC |
| 3:No known function | NM_199605 | zgc:66382 | CAGCCTCCAAATATGCAATACATCCATTTTCTTT |
| | | | GTTTTGGAGATAACACTTGTGAAAAT |
| 3:No known function | NM_200795 | zgc:73337 | GTGTGTTGGGTCCCTTGGTTTTAGATTGATTTTG |
| | | | AGGAAGAGAAGTCAAAGAATTCTTAC |
| 3:No known function | BG306034 | sb:cb62 | CAAGTGAAATGAGCGACTGTGTTTGTGAATATTT |
| | | | ATGCACATGCATTTTGTGTCCAACTG |
| 3:No known function | BI705018 | si:ch211-217k17.11 | GGTTTACCAAAAGAAATACCCAAAGTGTACGTTC |
| | | | AGGGAGATACCAGGATACACGTTTCA |
| 4:No annotation | BM181859 | no annotation | AACTACCAAATCTCACTTTGTAAAGGATTCACAC |
| | | | GATGACCACTAGAGGTCTATCCGCTT |
| 4:No annotation | BE606169 | no annotation | TACTAAGAGAAGTCATGCAGTGATGATTTCGCCG |
| | | | TGTACGTACCAAAGTAACACTGTTTG |
| 4:No annotation | AW076815 | no annotation | TGTAAAATTTTACAGAGGAAAGGCAAGTTCACTC |
| | | | AATAGCAAATTCCTCATTTACTCACC |
| 4:No annotation | BG308558 | no annotation | CTTTAAAGGCAAGACACTGCAGGCACCTGAGATT |
| | | | TCGGTCTTTTTAGCTTCTCATTCATT |
| 4:No annotation | AL924436 | no annotation | AATGGCGTATCGGTAACATGATGTCCAGATAGCT |
| | | | TCATTTCAACTGGAAACGATCAGCTG |
| 4:No annotation | AL925833 | no annotation | GTGAACTTTGCATTTGAAACCCAGCTTCTTAGCC |
| | | | AGCTCAGTGAGCAGATCCATACAGTA |
| 4:No annotation | BI475983 | no annotation | ATTTGTGCATATGACGTATGTAACCTCATAACCC |
| | | | TGAGGTTACGCATTTGACTTTGGCCA |
| 4:No annotation | BI845607 | no annotation | TCAAAGTGACCACAACTCCTTGCACAACATTACA |
| | | | GTGAGCAGTCTATACAAGTACATTTT |
| 4:No annotation | AI396694 | no annotation | TGTTTGAGGCCAGACTTTTTACTTTCATTTGAGA |
| | | | AAAATACAGTAGTCAGTATTTCAGCT |
| 4:No annotation | AJ286843 | no annotation | CTTTCATTTAGACATTAATCTGTCACAGTTCTCC |
| | | | AGGCAAGACGCAATGACCTCAGCACC |
| 4:No annotation | BQ264053 | no annotation | AATTGCCCACTTTGTATTTGGAGAGGCCACAAAC |
| | | | TTGCTTTTTTGGTTTGACCCAGTAAT |
| 4:No annotation | BQ092076 | no annotation | GCGACCACACATGAGCTGTACAGCACTGTTAAAG |
| | | | AAACACTCCACTTTTTATTTAGGAAA |
| 4:No annotation | TC269649 | no annotation | TCATTCAATTGTATGCTGCTCGTTATTCAGTTCA |
| | | | CTGGTATGTTTTATGTCTTGCTTCAA |
| 4:No annotation | AL909084 | no annotation | TTCATGTTCTCTGCACTTTAAATGGCAGAAGAAC |
| | | | TTGTCGTTTCAACCTTAATGTGGGTT |
| 4:No annotation | ENSDART00000048550 | no annotation | TATATGTGTGCTGCAAACCTGTGAGATCAAATGC |
| | | | AGTCATCCTGCTTCACAGTTTCATTG |
| 5:Control gene present in | ds-red control | ds-red control | TTCATGCGCTTCAAGGTGCGCATGGAGGGCTCCG |
| replicating entity | | | TGAACGGCCACGAGTTCGAGATCGAG |

TABLE 2
miRNAs that are up-regulated after yolk
injection of Mycobacterium marinum.

| | | response to |
|---|---|---|
| | | M. marinum |
| miR name | | yolk injec- |
| (mirbase) | mir sequence | tion |
| dre-miR-146b | ugagaacugaauuccaagggug | up |
| dre-miR-21 | uagcuuaucagacugguguuggc | up |
| dre-miR-29a | uagcaccauuugaaaucgguua | up |
| dre-miR-132 | uaacagucuacagccauggucg | down |
| dre-miR-132* | accguggcauuagauuguuacu | down |
| dre-miR-146a | ugagaacugaauuccauagaugg | up |
| (dre-miR-21*) | cgacaacagucuguaggcuguc | up |
| not yet | | |
| in mirbase | | |
| dre-miR-29b | uagcaccauuugaaaucagugu | up |
| dre-miR-196a | uagguaguuucauguuguuggg | (down low |
| | | concentration |
| | | only) |
| dre-miR-363 | aauugcacgguauccaucugua | (down low |
| | | concentration |
| | | only) |
| (dre-miR-143*) | ggugcagugcugcaucucuggu | down |
| not yet | | |
| in mirbase | | |
| dre-miR-217-1 | uacugcaucaggaacugauugg | (down low |
| | | concentration |
| | | only) |
| dre-miR-193b | aacuggcccgcaaagucccgcu | (down low |
| | | concentration |
| | | only) |
| dre-miR-212 | uaacagucuacagucauggcu | (down low |
| | | concentration |
| | | only) |
| dre-miR-365 | uaaugccccuaaaaauccuuau | down |
| dre-miR-455 | uaugugcccuuggacuacaucg | down |
| dre-miR-489 | agugacaucauauguacggcugc | (down low |
| | | concentration |
| | | only) |
| dre-miR-722 | uuuuuugcagaaacguuucaga | down |
| | uu | |
| dre-miR-34 | uggcagugucuuagcugguugu | (down low |
| | | concentration |
| | | only) |

The invention is herein exemplified using zebrafish and carp embryo injection. However, the system and method of the present invention are not limited to zebrafish embryos, but are also applicable to other in vivo models that represent externally viable embryos.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition of the invention may comprise additional component(s) than the ones specifically identified, said additional component(s) not altering the unique characteristics of the invention.

The word “approximately” or “about” when used in association with a numerical value (approximately 10, about 10) preferably means that the value may be the given value of 10 plus or minus 1% of the value.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

It will be clear that the above description and drawings are included to illustrate some embodiments of the invention, and not to limit the scope of protection. Starting from this disclosure, many more embodiments will be evident to a skilled person which are within the scope of protection and the essence of this invention and which are obvious combinations of prior art techniques and the disclosure of this patent.

### EXAMPLES

### Methods

### Fertilization

The fish eggs were fertilized according to standard protocols, e.g. using breeding tanks with dividers or in vitro fertilization techniques. At various stages after fertilization (outlined in FIG. 3), the embryos were transferred to a custom-made embryo holding device. The embryo holding device serves to hold the embryo in a fixed position during the intrayolk injection.

### Injection

The pathogens were suspended at a low density in carrier material. The standard carrier material was 2% polyvinyl pyrrolidone (PVP) in PBS. The pathogen suspension was transferred via back-loading to the capillary and the loaded capillary was then connected to the robotic micromanipulator via the capillary holder and to the injector via the tubing. The embryos were injected into the yolk.

### Transcriptome, Proteome, Metabolome Screening and Selection of High Throughput Marker Sets

Transcriptome screening: Zebrafish embryos were snap frozen in liquid nitrogen and RNA was isolated using the miRNAeasy kit (Qiagen). RNA concentrations were determined spectrophotometrically using the NanoDrop (Thermo Scientific).

For microarray analysis, zebrafish RNA-derived cDNA samples were labeled with Cy3 or Cy5 (GE Healthcare) using the Amino Allyl MessageAmp II aRNA Amplification kit (Ambion) and hybridized to custom-designed 4×44K zebrafish oligonucleotide microarrays (Agilent).

Proteome screening: Zebrafish embryos were ground with a pestle in liquid nitrogen in 1.5 mL Eppendorf tubes and vortexed for 30 seconds in a lysis buffer consisting of 9 parts 20 mM Tris-HCl, pH 8.5, 20 mM NaCl, 2% sodium deoxycholate and 1 part protease inhibitor cocktail (P 8340, Sigma-Aldrich). The samples were placed on a shaking table for at least 20 minutes at room temperature before spinning down cellular debris at 16,100×g for 10 minutes at 4° C. The supernatant was transferred to a fresh tube and treated with benzonase (E 1014, Sigma-Aldrich) to degrade the viscous DNA. The protein concentration in the extracts was measured and sample and protein extraction quality were checked by SDS-PAGE. An aliquot of each sample was digested and analyzed by LC-MS/MS, followed by deep proteome analysis using LC/LC-MS/MS.

In order to facilitate a better understanding of experimental implementation of the present invention below a description is provided of injection and screening experiments verified by our laboratory experiments.

### Injection of Replicating Entities in the Yolk of Zebrafish

It was investigated whether it was possible to design a system that only relies on fast injection and neglects accuracy combined with post-injection high throughput filtering for embryos that were not injected in a desired way. This is therefore a novel approach than reported in alternative injection systems for vertebrate embryos. Instead of accurately injecting embryos with a capacity of up to thousands per day per system, we explored the possibility of inaccurately injecting embryos with a capacity of up to ten thousands per day per system, combined with high throughput post-screening for accuracy. A report of the results of injection and subsequent read out is given for the examples of granuloma-inducing mycobacteria (Mycobacterium marinum), non-granuloma-inducing mycobacteria (Mycobacterium smegmatis, M. bovis), neuron-infecting mycobacteria (e.g. M. leprae), pathogenic gram-negative bacteria (Edwardsiella tarda), non-pathogenic lactobacilli, (Lactobacillus casei shirota, L. casei defensis, L. casei rhamnosus), non-pathogenic gram-negative bacteria (Rhizobium leguminosarum), non-pathogenic yeasts (Saccharomyces cerevisiae), pathogenic yeasts (Candida albicans), pathogenic fungi (Aspergillus fumigates), protists (Plasmodium berghei); Trypanososomes, (e.g. Trypanosoma carassii;), viruses (spring viremia of carp virus (SVCV)), vertebrate cancer cells such as from human (bone tumor cells).

### Example a

### The Effect of Drugs on the Response of Zebrafish Embryos to Intrayolk Injection with Mycobacterium marinum

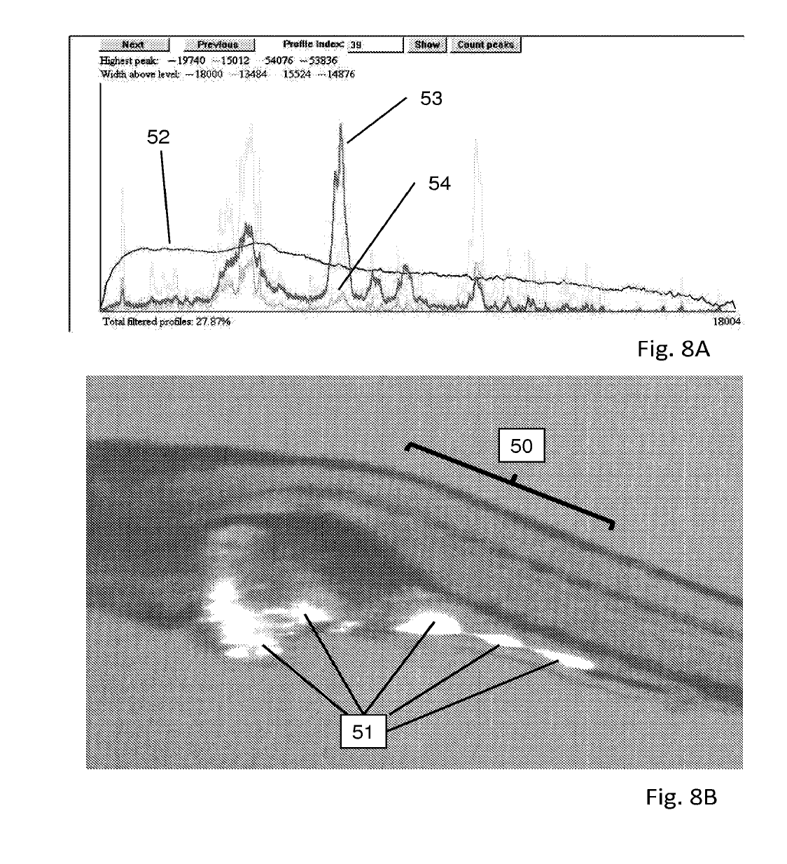

Mycobacterium marinum strain E11 stably expressing cherry fluorescent protein (CherryFP) was cultured in Middlebrook 7H9 medium plus 50 μg/ml hygromycin at 30° C. to an O.D.₆₀₀ₙₘ of ˜1.0. The culture (10 ml) was spun for 30 seconds at 13,000 rpm and the pellet was washed twice with PBS and then resuspended in 10 μl 2% PVP (PVP-40K in PBS), resulting in a density of ˜20,000 CFU/nl. The culture was diluted further in 2% PVP to 20 CFU/nl and 5 CFU/nl. Zebrafish eggs were fertilized by natural mating that was triggered by the removal of dividers in breeding tanks. Viable translucent embryos were selected using COPAS XL-mediated laser extinction profiling and sorted to custom-made 96-well embryo holders. The embryo holder was attached to an automated stage positioner (Märzhäuser MT mot. 200×100-1 mm MR) that was connected to a controller (Märzhäuser Tango2-desktop controller). Glass needles were pulled from borosilicate capillaries (Harvard Apparatus GC100TF-10; 1 mm outer diameter, 0.78 mm inner diameter) and back-loaded with Mycobacterium suspensions or carrier alone (optionally mixed with fluorescent dye; fluorescein at 0.1-1 mg/ml) using a microloader pipette (Eppendorf). The needle tip was clipped to a diameter of ˜15 μm and the needle was attached to a programmable micromanipulator (Eppendorf InjectMan NI2) and connected to a Femtojet Express injector (Eppendorf; settings: Pi=400 HPa, Ti ˜0.4 s) with external compressor (JUN-AIR 3-4). Mycobacterium suspension or carrier alone was injected automatically into the yolk of 16- to 256-cell stage zebrafish embryos by programming the repetitive activities of the stage positioner controller, the micromanipulator and the Femtojet injector via RS232 ports from a Linux PC using a custom-made Python script. On the basis of the CherryFP and fluorescent dye content, only embryos that contained the proper amount of injected microbes or carrier were selected using COPAS XL-mediated fluorescence profiling, sorted to 96-well microplates and incubated at 28° C. At 2 days post injection (2 dpi) the embryos were exposed to ethambutol (2 mM in water), generic H89 (10 μM in 0.5% DMSO; Kuijl et al., 2007) or 0.5% DMSO alone. The drugs were refreshed daily. At 5 dpi the larvae were automatically screened for normal development and the presence of CherryFP-labeled granulomas by COPAS XL-mediated laser extinction and fluorescence profiling (FIG. 8). Granulomas were present in 97% of the larvae that were incubated with DMSO alone, whereas the larvae that were treated with ethambutol or H89 did not contain granulomas. The results of the COPAS XL screen were confirmed by visual inspection using routine stereo fluorescence microscopy. Subsequently, the larvae were snap frozen in liquid nitrogen and ground into a powder, one half of which was used for RNA isolation and the other half for protein extraction. Total RNA was isolated using the miRNAeasy kit (Qiagen) and the RNA profile of the injected larvae was compared with that of uninjected larvae using microarray analysis and whole mRNAseq (according to the standard Illumina protocol). For microarray analysis, RNA-derived cDNA samples were labeled with Cy3 or Cy5 (GE Healthcare) using the Amino Allyl MessageAmp II aRNA Amplification kit (Ambion) and hybridized to custom-designed 4×44K zebrafish oligonucleotide microarrays (Agilent). Results of the microarray analyses are described below. For whole mRNAseq analysis, the RNA-derived cDNA was sequenced using the Illumina GAIIx sequencer. Protein was extracted from the other half using 20 mM Tris-HCl pH8.5, 20 mM NaCl, 2% Na-deoxycholate in the presence of 10% protease inhibitor cocktail (Sigma P8340). The results of the drug screen showed that the yolk injection method is suitable for the screening of antimicrobial drugs.

### Example b

### The Response of Carp Embryos to Intrayolk Injection with Mycobacterium marinum

The conditions were identical to the description in example (a) with the following exceptions. Carp embryos were obtained via in vitro fertilization and treated with pineapple juice to remove stickiness. Intrayolk injection was performed using one day-old carp embryos after manual dechorionation. The infected carp embryos were studied using stereo microscopy and confocal laser scanning microscopy (Zeiss Observer, inverted CLSM). The results show clear granuloma formation in the body of the fish, e.g. in tail fins, blood island and brain areas. These results were highly similar as found with zebrafish yolk injection of Mycobacterium marinum strains. The size of the carp larvae at 5 dpi (˜7 mm length) allowed analysis in the COPAS XL Biosorter. The response of the carp embryos to intrayolk injection with Mycobacterium marinum was determined via total RNAseq on an Illumina GAIIx sequencer. Full sequencing of the transcriptome was performed at 5 days using an Illumina GAIIx sequencing system. The results were compared with the transcriptome data of zebrafish after yolk injection using the same conditions.

### Response Analysis to Different Treatments

### Example c

### The Response of Zebrafish Embryos to Intrayolk Injection with Mycobacterium leprae

The conditions were identical to the description in example (a) with the following exceptions. The whole procedure of microbe preparation, intrayolk injection and harvesting of the zebrafish embryos was carried out at MLIII safety level. Mycobacterium leprae was labeled with Dylight-red 654/673 (Pierce) prior to injection. The zebrafish embryos were manually injected with 6, 30 or 60 CFU/nl live or dead Mycobacterium leprae bacteria. The survival and spread of the M. leprae was studied using confocal laser scanning microscopy (Zeiss Observer). The results showed that M. leprae was able to survive until the end stage of the experiment (5 dpi) and was present in many regions in the body: inside blood vessels, inside presumptive immune cells, close to the gut area and close to the gill area.

### Example d

### The Response of Zebrafish Embryos to Intrayolk Injection with Mycobacterium bovis (Bacillus Calmette-Guérin (BCG-P3))

The conditions were identical to the description in example (a) with the following exceptions. Unlabeled bacteria were used.

### Example e

### The Response of Zebrafish Embryos to Intrayolk Injection with Mycobacterium smegmatis

The conditions were identical to the description in example (a) with the following exceptions. Unlabeled bacteria were used.

### Example f

### The Response of Zebrafish Embryos to Intrayolk Injection with Rhizobium leguminosarum Strain RBL5523

The conditions were identical to the description in example (a) with the following exceptions. Bacterial suspensions were directly obtained from cultures on plate, washed in PBS and resuspended in 2% PVP-40 in PBS.

### Example g

### The response of zebrafish embryos to intrayolk injection with Lactobacillus casei shirota (Yakult)

The conditions were identical to the description in example (a) with the following exceptions. The bacterial culture was bought in a grocery store, washed several times with PBS and resuspended in 2% PVP-40K in PBS. Unlabeled bacteria were used.

### Example h

### The response of zebrafish embryos to intrayolk injection with Trypanosoma carassii

The conditions were identical to the description in example (a) with the following exceptions. The Trypanosoma culture (6 ml at ˜10⁸/ml) was centrifuged and the pellet resuspended in 10 μl PVP-40K in PBS. This concentrated suspension was further diluted 1:10 and 1:100 in 2% PVP-40K.

### Example i

### The Response of Zebrafish Embryos to Intrayolk Injection of Plasmodium berghei

Plasmodium berghei sporozoites were isolated with a microneedle from mosquitoes that were blood fed from infected mouse. The salivary gland of the mosquitoes was excised under a stereo microscope (Leica). The parasites were sucked up from the 4 long lobes of the salivary glands using an Eppendorf Cell-Tram oil-based micro-needle system. In the second step the parasites were injected with the same needle into the yolk of embryos. Plasmodium merozoites were obtained from blood of infected mice. The isolated infected red blood cells were injected into the yolk. As a control uninfected red blood cells were tested. For comparison, a part of the bug's lobe was implanted manually. In both methods, Plasmodium was shown to survive for over three days after injection as confirmed by confocal laser scanning microscopy.

### Example j

### The Response of Zebrafish Embryos to Intrayolk Injection with Tumor Cells

The conditions were identical to the description in example (a) with the following exceptions. Unlabeled SJSA osteosarcoma cells were used. Zebrafish embryos at the earliest stages after fertilization were injected with 5-800 cells.

### Example k

### The Response of Zebrafish Embryos to Intrayolk Injection with Saccharomyces cerevisiae

The conditions were identical to the description in example (a) with the following exceptions. Normal baker's yeast was obtained from Unilever, the Netherlands, and resuspended in PBS.

### Example 1

### The Response of Zebrafish Embryos to Intrayolk Injection with Candida albicans

The conditions were identical to the description in example (a) with the following exceptions. Candida was grown at 30 degrees Celsius in YPD medium in the yeast phase at which stage they are not sticking together. Optimal pH for yeast growth was pH 4.

### Example m

### The Response of Zebrafish Embryos to Intrayolk Injection with Edwardsiella tarda

The conditions were identical to the description in example (a) with the following exceptions. A liquid culture of E. tarda was grown on TSA medium plates overnight and bacteria were scraped off and suspended in PBS to an OD of 0.3. The suspension was diluted so that an injection of 2 CFU was reached. Since this injection is potentially lethal we stopped the experiment at earlier stages than in the other injections listed here.

### Example n

### The Response of Zebrafish Embryos to Intrayolk Injection with Aspergillus niger

The conditions were identical to the description in example (a) with the following exceptions. A black suspension of spores at a concentration of 7×10⁷ were spun down and concentrated in PBS.

### Example o

### The Response of Sea Squirt Embryos to Intrayolk Injection with Mycobacterium marinum

To demonstrate the method of the invention in sea squirts, embryos at different time points post fertilization were injected with fluorescently-labeled Mycobacterium marinum bacteria at a dose of 50-200 colony forming units. Sea squirt colonies were collected in the province of Zeeland, the Netherlands, and kept in sea water aquaria for several days. Embryos were harvested manually. Microscopy was performed using confocal laser scanning microscopy (Leica SPE). It was concluded that the sea squirt embryos and the bacteria were still viable after injection.

### Microarray Analysis

Samples obtained from zebrafish embryos injected with the above mentioned examples were analyzed on zebrafish Agilent microarray chips as described previously (Stockhammer et al, 2009). The data was analyzed using the software program Rosetta Resolver and normalized data sets were exported into Microsoft Excel. Gene transcripts that responded to injection with a P value of smaller than 10⁻⁵ were used for comparisons. Comparison of all the data sets led to the identification of gene sets that were characteristic for particular treatments. The genes were divided into categories: category 1 are genes that were specific for one particular treatment, category 2 are genes that were common for a particular group of treatments and category 3 are genes that were never responding to any treatment (control genes). These gene sets were subsequently divided into sub categories: a) genes of which there are indications for their function, b) genes of which there is no known indication of function yet described, c) Genes of which there was no prior evidence of expression. Subsequently we checked whether these genes had a homolog in other fish species like carp fish. Using our method we have identified many genes of sub-category b and c showing that we can use our high throughput method to identify new marker genes involved in disease processes.

Transcriptomic changes in embryos were assayed using custom zebrafish microarrays (Agilent Technologies). A subset of the micro array probes based on criteria mentioned in the text was annotated in great detail and design was towards probes that are common for all fish species. For each of seven infection types, between two and six biological replicates were analysed (26 samples in total).

Initial data processing (normalization and fold change calculation) was performed using the Rosetta Resolver software. Probes highly specific for one or more infections were selected using K-means clustering on fold changes (MeV software, www.tm4.org). The heatmap (FIG. 9) shows the average change in detection for the 94 genes listed in Table 2 (rows), averaged over the different samples per condition (columns). Black indicates decreased expression, white increased expression. Because of some redundancy in the probe/gene mapping, the actual number of probes shown here is 113.

### REFERENCES

Brannon, M. K., J. M. Davis, J. R. Mathias, C. J. Hall, J. C. Emerson, P. S. Crosier, A. Huttenlocher, L. Ramakrishnan, and S. M. Moskowitz. 2009. Pseudomonas aeruginosa Type III secretion system interacts with phagocytes to modulate systemic infection of zebrafish embryos. Cell Microbiol. 11, no. 5:755-768.
Cabrita, E., V. Robles, P. Herráez. (Editors) 2009. Methods in reproductive aquaculture. CRC Press.
Davis, J. M. and L. Ramakrishnan. 2009. The role of the granuloma in expansion and dissemination of early tuberculous infection. Cell 136, no. 1:37-49.
Fletcher, G. L., M. A. Shears, E. S. Yaskowiak, M. J. King and S. V. Goddard. 2004. Gene transfer: potential to enhance the genome of Atlantic salmon for aquaculture. Aust. J. Exp. Agr. 44, 1095-1100.
Heasman, J. 2002. Morpholino oligos: making sense of antisense? Dev. Biol. 243, no. 2: 209-14.
Lawson, N. D. and B. M. Weinstein. 2002. In vivo imaging of embryonic vascular development using transgenic zebrafish. Dev.Biol. 248, no. 2:307-318.
Lee, L. M., E. A. Seftor, G. Bonde, R. A. Cornell, and M. J. Hendrix. 2005. The fate of human malignant melanoma cells transplanted into zebrafish embryos: assessment of migration and cell division in the absence of tumor formation. Dev. Dyn. 233, no. 4:1560-1570.
Levraud, J. P., P. Boudinot, I. Colin, A. Benmansour, N. Peyrieras, P. Herbomel, and G. Lutfalla. 2007. Identification of the zebrafish IFN receptor: implications for the origin of the vertebrate IFN system. J. Immunol. 178, no. 7:4385-4394.
Marques, I. J., F. U. Weiss, D. H. Vlecken, C. Nitsche, J. Bakkers, A. Lagendijk, L. I. Partecke et al. 2009. Metastatic behaviour of primary human tumours in a zebrafish xenotransplantation model. BMC. Cancer 9, no. 1:128.
Meijer, A. H., A. M. van der Sar, C. Cunha, G. E. Lamers, M. A. Laplante, H. Kikuta, W. Bitter, T. S. Becker, and H. P. Spaink. 2008. Identification and real-time imaging of a myc-expressing neutrophil population involved in inflammation and mycobacterial granuloma formation in zebrafish. Dev. Comp Immunol. 32, no. 1:36-49.
Paschos, I., L. Natsis, C. Nathanailides, I. Kagalou, and E. Kolettas. 2001. Induction of gynogenesis and androgenesis in goldfish Carassius auratus (var. oranda). Reprod Domest Anim. 36, no. 3-4:195-8.
Prajsnar, T. K., V. T. Cunliffe, S. J. Foster, and S. A. Renshaw. 2008. A novel vertebrate model of Staphylococcus aureus infection reveals phagocyte-dependent resistance of zebrafish to non-host specialized pathogens. Cell Microbiol. 10, no. 11:2312-2325.
Recoubratsky, A. V., B. I. Gomelsky, O. V. Emelyanov, and E. V. Pankratyeva, 1992. Aquaculture 108, 13-19.
Renshaw, S. A., C. A. Loynes, D. M. Trushell, S. Elworthy, P. W. Ingham, and M. K. Whyte. 2006. A transgenic zebrafish model of neutrophilic inflammation. Blood 108, no. 13:3976-8.
Stockhammer, O. W., A. Zakrzewska, Z. Hegedus, H. P. Spaink, and A. H. Meijer. 2009. Transcriptome profiling and functional analyses of the zebrafish embryonic innate immune response to Salmonella infection. J. Immunol. 182, no. 9:5641-5653.
Takagi S., T. Sasado, G. Tamiya, K. Ozato, Y. Wakamatsu, A. Takeshita, M. Kimura. 1994. An efficient expression vector for transgenic medaka construction. Mol Mar Biol Biotechnol. 3, no. 4:192-199.
Thai, B. T., and T. G. Ngo. 2004. Use of pineapple juice for elimination of egg stickiness of common carp (Cyprinus carpio L.). Asian Fisheries Science 17: 159-162.
Van der Sar, A. M., R. J. Musters, F. J. van Eeden, B. J. Appelmelk, C. M. Vandenbroucke-Grauls, and W. Bitter. 2003. Zebrafish embryos as a model host for the real time analysis of Salmonella typhimurium infections. Cell Microbiol. 5, no. 9:601-611.
White, R. M., A. Sessa, C. Burke, T. Bowman, J. LeBlanc, C. Ceol, C. Bourque et al. 2008. Transparent adult zebrafish as a tool for in vivo transplantation analysis. Cell Stem Cell 2, no. 2:183-189.
Zappe, S., M. Fish, M. P. Scott, and O. Solgaard. 2006. Automated MEMS-based Drosophila embryo injection system for high-throughput RNAi screens. Lab Chip. 6, no. 8:1012-1019.

1. A method for screening chemical compounds or compositions in an embryo or larval system, comprising the steps of:
(a) providing a plurality of start biosystems comprising living eggs or embryos, comprising a yolk, of aquatic developing chordates which are at a stage prior to 22 hours post fertilization;
(b) introducing one or more replicating entities into the yolks of at least a set of said start biosystems;
(c) exposing a set of said start biosystems to said chemical compounds or compositions;
(d) allowing said start biosystems to develop into a plurality of embryos or larvae;
(e) determining a response in said embryos or larvae, and
(f) correlating said response with said chemical compounds or compositions.

2. The method of claim 1, wherein the start biosystems are at a stage of up to a 128 cell blastula.

3. The method of claim 1, wherein said start biosystems are said embryos of aquatic developing chordates.

4. The method of claim 3, wherein said aquatic developing chordates are fish.

5. The method of claim 1, wherein said one or more replicating entities are bacteria, fungi, yeasts, protists, or a combination thereof.

6. The method of claim 1, wherein said one or more replicating entities comprise cancer cells or clusters of cancer cells.

7. The method of claim 1, wherein said one or more replicating entities comprise viruses.

8. The method of claim 1, wherein said one or more replicating entities have a volume of less than about 3 nanoliters preferably less than about 2 nanoliters.

9. The method of claim 1, wherein said introducing is by injection.

10. The method of claim 9, wherein said injection is via a needle or ballistic delivery.

11. The method of claim 1, wherein said exposing step (c) comprises introducing said chemical compounds or compositions into the yolk.

12. The method according to claim 1, wherein said exposing step (c) is performed simultaneously with said introducing step (b).

13. The method of claim 1, wherein said exposing step (c) is performed after said introducing step (b).

14. The method of claim 1, which is for determining a mechanism underlying an effect produced by one or more of said chemical compounds or compositions, which method further comprises a step of
(g) introducing a gene-function-modifying molecule into the yolk.

15. The method of claim 14, wherein step (g) is performed simultaneously with step (b) and/or step (c).

16. The method of claim 14, wherein the gene-function-modifying molecule is a gene-silencing molecule.

17. The method of claim 1, wherein said plurality of start biosystems are provided as a flow through system.

18. The method of claim 1, wherein said plurality of start biosystems are provided as a holding system in which said start biosystems are retained at substantially fixed positions.

19. The method of claim 1, wherein said replicating entities are introduced in at least about 300 start biosystems per hour or in at least about 1500 start biosystems per hour.

20. The method of claim 1 adapted for high throughput screening of said chemical compounds or compositions, wherein:
(1) step (a) comprises positioning an array of a plurality of said start biosystems in a holder in which said embryos are retained at their position;
(2) step (b) comprises injecting said one or more replicating entities into the yolk of said plurality of said start biosystems in said holder.

21. The method of claim 1, wherein said replicating entities are introduced in the presence of carrier compounds.

22. The method of claim 21, in which the replicating entities are embedded in carrier material selected from the group consisting o
(a) an inert non-immunogenic fluid such as polyvinylpyrrolidone (PVP),
(b) an inert non-immunogenic solid polymer such as cellulose sulfate, chitin, chitosan or plastic,
(c) an inert non-immunogenic solid photo-degradable polymer such as a plastic, and
(d) a hydrogel.

23. The method of claim 1, wherein said response is measurable at a physical level, at a transcriptome level, at a proteome level or at a metabolome level.

24. The method of claim 23, wherein said response is measured optically.

25. A method for determining a mechanism responsible for an effect of functional chemical compounds or compositions on disease development in an embryonic or larval system, comprising the steps of:
(a) providing a plurality of start biosystems comprising living eggs or embryos, comprising yolks, of aquatic developing chordates which are at a stage prior to 22 hours post fertilization;
(b) introducing one or more replicating entities capable of inducing disease development into the yolks;
(c) exposing said set of said start biosystems to said functional chemical compounds or compositions;
(d) exposing a subset of said start biosystems to a gene-function-modifying molecule;
(e) allowing said start biosystems to develop into a plurality of embryos or larvae;
(f) determining a response in said embryos or larvae,
(g) correlating said response with said gene-function-modifying molecules, and
(h) identifying gene-function-modifying molecules that counteract the effect of said functional chemical compounds or compositions on said disease development.

26. The method of claim 25, wherein said gene-function-modifying molecule is a gene-silencing molecule.

27. The method of claim 25, wherein said functional chemical compounds or compositions inhibit, slow or halt disease development.

28. The method of claim 25, wherein steps (b), (c) and (d) are performed simultaneously.

29. A high throughput system for screening a set of chemical compounds or compositions using a plurality of start biosystems having a yolk, said biosystems comprising living eggs or living embryos of aquatic developing chordates, said system comprising:
(a) a controller, comprising a memory;
(b) a transporter, operationally coupled to said controller, for passing start biosystems individually past an introduction position;
(c) an injector, operationally coupled to said controller, adapted for introducing into yolks a living replicating entity in a set of said start biosystems at said introduction position;
(d) an exposure system for exposing a set of said start biosystems to said chemical compounds or compositions, said exposure system being operationally coupled to said controller;
(e) a first detector, operationally coupled to said controller, for measuring a first response of said start biosystems and transmitting the measurements of said first response to said controller which stores measurements coupled to identities of the introduced replicating entity and the chemical compound or composition to which the start biosystems were exposed.

30. The system of claim 29, wherein said transporter comprises a holder comprising at least one cavity, dimensioned for holding one of said start biosystems in a substantially fixed position.

31. The system of claim 29, wherein said transporter is adapted for passing at least 300 start biosystems or at least 1500 start biosystems per hour past said introduction position, in an embodiment at least 1500 start biosystems per hour.

32. The system of claim 30, wherein said transporter comprises an actuator for displacing said holder for passing said start biosystems individually past said introduction position.

33. The system according to claim 29, further comprising
(f) an additional detector, operationally coupled to said controller, for identifying an additional property of each of said start biosystems, wherein an identifier for said additional property is stored in said controller memory.

34. The system according to claim 29, further comprising
(g) a biological safety cabinet confining said transporter and said injector, said safety cabinet preferably complying with at least to the biosafety level 2 requirements, preferably with the biosafety level 3 requirements.

35. The system according to claim 29, wherein said transporter comprises a holder comprising a plurality of cavities regularly spaced, the size of each cavity being adapted for holding one starting biosystem at a substantially fixed position.

36. The system according to claim 35, wherein said holder comprises a cover slide with an injection port through holes positioned at said cavities that
(i) prevents said start biosystems from escaping from said cavities, and
(ii) allows said injector to deliver said replicating entity into said yolk.

37. The system according to claim 29, wherein said transporter comprises a groove in which said start biosystems are spaced regularly and situated side-by-side, optionally comprising a cover slide with a slit positioned at said groove that prevents said start biosystems from escaping from said groove, wherein said slit is preferably dimensioned to allow said injector to deliver a replicating entity into said yolk.

38. The system according to claim 29, wherein said transporter comprises a flow-through channel.

39. The system according to claim 29, comprising a rotating disc with cavities around its circumference, each cavity for holding a start biosystem.

40. The system according to claim 29, wherein said transporter comprises at least one cavity for holding a start biosystem, said cavity coupled to a pressured channel debouching in said cavity for holding a start biosystem at a substantially fixed position in said cavity during operation.

41. A method for identifying a marker gene, a marker protein or a marker metabolite characteristic of a specific disease or condition, said method comprising the steps of:
(a) providing a plurality of start biosystems comprising living eggs or embryos, which comprise yolks, of aquatic developing chordates which are at a stage prior to 22 hours post fertilization;
(b) introducing one or more replicating entities capable of effecting said specific disease or condition into the yolk of at least a set of said start biosystems;
(c) determining a transcriptome, proteome or metabolome in at said set of start biosystems;
(d) comparing the transcriptome, proteome or metabolome of the biosystems in which replicating entities have been introduced with the transcriptome, proteome, or metabolome in biosystems into which no replicating entities have been introduced; and
(e) identifying a marker gene, marker protein or marker metabolite for said specific disease or condition.

42. (canceled)

43. A marker gene or set of marker genes identified by the method of claim 41 that can distinguish the effects of injection of replicating entities selected from the group consisting of Mycobacteria, probiotic lactobacilli, trypanosomes, cancer cells, yeasts, fungi, gram negative bacteria, and viruses.


SEQUENCE LISTING (112 sequences)

SEQ ID NO: 1 - DNA, 60 bp, Danio rerio
caagtttgcaatgatctcagctgggctgattttactctttaaatgtgagaatgctctctt

SEQ ID NO: 2 - DNA, 60 bp, Danio rerio
agcagtgaccaatcagacatatctactgtgagtgtcaacagtgaagaaagaggtgtgagt

SEQ ID NO: 3 - DNA, 60 bp, Danio rerio
cggcccatccgacgatattgcacttttgaacaaaatcaaggagcacacaattatacctat

SEQ ID NO: 4 - DNA, 60 bp, Danio rerio
gaccattaaagctggagatccaaacggatacgaccagctgctgttcttcaggaaggagac

SEQ ID NO: 5 - DNA, 60 bp, Danio rerio
tatgaaatacccaatgattcgcaatgtgagggagggtctgcacagagtcgagcaagaatt

SEQ ID NO: 6 - DNA, 60 bp, Danio rerio
agtgttttgctgtggatcagttcaggcatggacagaagaaaaacatacaagaagattcac

SEQ ID NO: 7 - DNA, 60 bp, Danio rerio
tgtctggacccctctgctccatgggttaagaagatcattgataggatcattgtcaagtaa

SEQ ID NO: 8 - DNA, 60 bp, Danio rerio
tcatgagcacgttgacaagcctctatcttggcaagaacggctgaatattatcaaaggcac

SEQ ID NO: 9 - DNA, 60 bp, Danio rerio
accgactgccaccatcaaactttgccaacttcttttgtgccatttatcagattaatttct

SEQ ID NO: 10 - DNA, 60 bp, Danio rerio
tcataggcacatgagacgggatgttaggcatatttgtccgtcagctttaccatggtctta

SEQ ID NO: 11 - DNA, 60 bp, Danio rerio
cattcttctattaccagactgatccatggctaacacacaagtggaaagatgagaagaaag

SEQ ID NO: 12 - DNA, 60 bp, Danio rerio
aacagttctgagaaaacttttttcaaagattcaaaagcagtgaacagagttggtctgctt

SEQ ID NO: 13 - DNA, 60 bp, Danio rerio
tggtaccaaactgacagcagtgagtctaatgttacctgatgactactgaaagaagacctg

SEQ ID NO: 14 - DNA, 60 bp, Danio rerio
gcacttctacccatggttgaaaatgataacggaaagaccttctttggggtgtttggtttg

SEQ ID NO: 15 - DNA, 60 bp, Danio rerio
gactaaggctaagaggcctccctgcatcgtgatgacttgttttatatgtaaaacaaagcc

SEQ ID NO: 16 - DNA, 60 bp, Danio rerio
ttgacttcccaattccagccaacaaaattatgtttgttgcgaggacgtcttcacgttgcg

SEQ ID NO: 17 - DNA, 60 bp, Danio rerio
acagcaaggtcagcaggaactagctgaattgaacacaaggtgaacaccaaaaacagcaga

SEQ ID NO: 18 - DNA, 60 bp, Danio rerio
tgaagatgtctgtctgttggcaatgaaaagagagcacaggaggtcaagagtgggaattct

SEQ ID NO: 19 - DNA, 60 bp, Danio rerio
gaagagatacgaggagctttctgacatcttctcagagaaagacaactattctcagagccg

SEQ ID NO: 20 - DNA, 60 bp, Danio rerio
tcttattgcacagtgtgttggttcactatctacttaaacatcttgatacagtaaaatgtt

SEQ ID NO: 21 - DNA, 60 bp, Danio rerio
gcgtttgaggcatgtatgcgctgtatgttgatgtagatctgcagtgtttgatcgtgagcg

SEQ ID NO: 22 - DNA, 60 bp, Danio rerio
aatactggagtgaaatgttacaagtttttctctcagtcggttagctggatcacagcagag

SEQ ID NO: 23 - DNA, 60 bp, Danio rerio
catgtttcggctctacgctccaggaggatggtgggaggatcactgactgcttctgtgccc

SEQ ID NO: 24 - DNA, 60 bp, Danio rerio
ggtctcttccacaccaaacacatcaatcacgacctcttccagcagcctgctgttctccag

SEQ ID NO: 25 - DNA, 60 bp, Danio rerio
gacacgatctatgtcaacgcaatggctttaaaagattttgaaaattcaagccacacatga

SEQ ID NO: 26 - DNA, 60 bp, Danio rerio
atcactgtacaggggaaagtctccactccgaaagttcatgtgtacagtcattttccagga

SEQ ID NO: 27 - DNA, 60 bp, Danio rerio
aggaacgcaaggatcgtgtagatatgacccatcccagcgtgcagcaaactgtacttctta

SEQ ID NO: 28 - DNA, 60 bp, Danio rerio
ctgaacaaatccagtagatttcattctctctttattaggggacttctattacaacaaacc

SEQ ID NO: 29 - DNA, 60 bp, Danio rerio
aagagcaagaagatctgaagcgtttcaccatcactctatttaaataaagctctgactgac

SEQ ID NO: 30 - DNA, 60 bp, Danio rerio
atgaaatttaattggatttcctactattggtcatcgattaaacggattaaacatcccggg

SEQ ID NO: 31 - DNA, 60 bp, Danio rerio
aatctcctttttcttggctcaaacagatcgaatggagctcgacgaggaacaaaaagcagc

SEQ ID NO: 32 - DNA, 60 bp, Danio rerio
atgtctttccttttgagatggaaaatggtacagaaccctatggcacagatgtgggcagct

SEQ ID NO: 33 - DNA, 60 bp, Danio rerio
agacagctaatagacagcaacaacaaggctaagtttgaactcgcagtgaaaatctattat

SEQ ID NO: 34 - DNA, 60 bp, Danio rerio
ctgcgcaaactctctacagtggcgtatcaggaagaggatcgcaaacgacttgaagcgctc

SEQ ID NO: 35 - DNA, 60 bp, Danio rerio
aaagagtgattggtagatgcctgcccatgtacagcatgtaatatggttctatttttcttg

SEQ ID NO: 36 - DNA, 60 bp, Danio rerio
acacgaggcttatgtacagtatgtctttgcatagtttaggatgcatcagtgtttcttatg

SEQ ID NO: 37 - DNA, 60 bp, Danio rerio
cactgatatccggaaagtcagagcttttacctttacatcaaggcattataatcatgatac

SEQ ID NO: 38 - DNA, 60 bp, Danio rerio
atgaagaaccgaacatcctcacgtcagtgtgcaaactgtttatacagatggaatcgcctc

SEQ ID NO: 39 - DNA, 60 bp, Danio rerio
gtccacgccgtaaacgatgtctactagctgttcgtgtccttgtgacgtgagtagcgcagc

SEQ ID NO: 40 - DNA, 60 bp, Danio rerio
tccctgtcatatcgaactccagacagcccttgacaagatcactaaatcacagcagaaact

SEQ ID NO: 41 - DNA, 60 bp, Danio rerio
ctgtgagatcaaatgcagtcatcctgcttcacagttacattgattttactacattttctt

SEQ ID NO: 42 - DNA, 60 bp, Danio rerio
acctcaatgggctatatgtgtgctgcaaacctgtgagatcaaatgcagtcatcctgcttc

SEQ ID NO: 43 - DNA, 60 bp, Danio rerio
tccacgaaacctctgtgaaattcagtggctccacaaatactcactttccacatctttaag

SEQ ID NO: 44 - DNA, 60 bp, Danio rerio
gctcccagaaatgtgtagatttatctgcatattatgaaagccttgtgataggctgagaac

SEQ ID NO: 45 - DNA, 60 bp, Danio rerio
tttgaccacttgttgctatatcatgttgcacttggttagagtacagttttatgctgaaat

SEQ ID NO: 46 - DNA, 60 bp, Danio rerio
gttacagaacaactctaactctctgagtcagaagaaactggagctggagaacagagtcac

SEQ ID NO: 47 - DNA, 60 bp, Danio rerio
ggtttgtcgatatggtcaacagcatgtcaataaaaacaaacctaaaaccacttcaaaaaa

SEQ ID NO: 48 - DNA, 60 bp, Danio rerio
gaccatcactgcaaactaaatcaccaagctaatgttcatggtcataatgttcatcaataa

SEQ ID NO: 49 - DNA, 60 bp, Danio rerio
ttggctaatgatagttcagaaataaacccctagccgatctcatgaaccggtcacaaatgt

SEQ ID NO: 50 - DNA, 60 bp, Danio rerio
catctgcaacagggaatataggcctgtatgtggtacagatggaattacgtacccaaacga

SEQ ID NO: 51 - DNA, 60 bp, Danio rerio
tcgtctgcatcctcgtgccgtcaactgcctgaagaagaagtgcggacacaccaacaacct

SEQ ID NO: 52 - DNA, 60 bp, Danio rerio
tctctgtccatcagagccggagtggtttcaactgttgatctcttagtggtcttattgaga

SEQ ID NO: 53 - DNA, 60 bp, Danio rerio
ctctgcttgatgcttcaacactgcataaatcatctcctctgtgtgtgctttgtatgcgct

SEQ ID NO: 54 - DNA, 60 bp, Danio rerio
ctgaacatatgctgcctactatttcatgctataccagggctgattcgagacatttggagg

SEQ ID NO: 55 - DNA, 60 bp, Danio rerio
atgacgattcagcacaactcatctcttgaggacttttatccgtaggcacacttctttatg

SEQ ID NO: 56 - DNA, 60 bp, Danio rerio
aacatcgaaatgcttcacgtctaagtctcgaagcagccctgcctggccttctcggtgggt

SEQ ID NO: 57 - DNA, 60 bp, Danio rerio
taaccagcaggtaccagcctggtttggtcctggttctggacccatatggctggatgaggt

SEQ ID NO: 58 - DNA, 60 bp, Danio rerio
gttcatcacctgctcggctctcaaaagattatgtgacactctttatccagactgtggagc

SEQ ID NO: 59 - DNA, 60 bp, Danio rerio
tggacgtggcctgttggctgattcttgtctaccgccgaattaacaaattggtcattctaa

SEQ ID NO: 60 - DNA, 60 bp, Danio rerio
tcataaccaagtttatgcaaaacttggtgacaaggtagatggagtgctttgtgatgggcc

SEQ ID NO: 61 - DNA, 60 bp, Danio rerio
tagacactgacatgctcgggaacagtggagaagcggatcatgattgtcttgtgggtcgag

SEQ ID NO: 62 - DNA, 60 bp, Danio rerio
agattttgttagacctggtccagtcaagtactgcatacagaatattacaaagccttctag

SEQ ID NO: 63 - DNA, 60 bp, Danio rerio
ttgctcttttcctctcttattttgtgcatgagcaacaacattgatgaaataatgtttgcg

SEQ ID NO: 64 - DNA, 60 bp, Danio rerio
cctttctttgacatatttgcttaattcttaatatgttatattgaagttaaagtggtgtta

SEQ ID NO: 65 - DNA, 60 bp, Danio rerio
aggcttcaatttgtcatcggcactacttgattcttgcgtgaaacttataaatgtatattg

SEQ ID NO: 66 - DNA, 60 bp, Danio rerio
gtctatacctgagaaatgacacaaacgccgttaagcaggggtgagtatttgcgcttgatt

SEQ ID NO: 67 - DNA, 60 bp, Danio rerio
gttagcatacttctttaaccatgacgatcttgtactcgcgtacagaactgtcagtgatta

SEQ ID NO: 68 - DNA, 60 bp, Danio rerio
cacgggttgagaatggttcaattcccatggtgtcgacatgctaacgtagagttgtctacg

SEQ ID NO: 69 - DNA, 60 bp, Danio rerio
gagaactcgtaatattgtgatttactcgatggtgtaatactgcactagtgctgtgcgagt

SEQ ID NO: 70 - DNA, 60 bp, Danio rerio
ttctaggccgctcagtggaaaagtgactcacactttcctattaataaacagtccttgcag

SEQ ID NO: 71 - DNA, 60 bp, Danio rerio
gaaatgtttcaggatcttcgtgggtctcttcagaaagtgagtcatttcctgcagtgcaca

SEQ ID NO: 72 - DNA, 60 bp, Danio rerio
tccccacctaaatttaaacctatattctgttctccgacagattgatttggttcaattatc

SEQ ID NO: 73 - DNA, 60 bp, Danio rerio
acagtgcttcaattttagtgggaacatttagtggacgtaaatttcagtaccgactggacc

SEQ ID NO: 74 - DNA, 60 bp, Danio rerio
cagcctccaaatatgcaatacatccattttctttgttttggagataacacttgtgaaaat

SEQ ID NO: 75 - DNA, 60 bp, Danio rerio
gtgtgttgggtcccttggttttagattgattttgaggaagagaagtcaaagaattcttac

SEQ ID NO: 76 - DNA, 60 bp, Danio rerio
caagtgaaatgagcgactgtgtttgtgaatatttatgcacatgcattttgtgtccaactg

SEQ ID NO: 77 - DNA, 60 bp, Danio rerio
ggtttaccaaaagaaatacccaaagtgtacgttcagggagataccaggatacacgtttca

SEQ ID NO: 78 - DNA, 60 bp, Danio rerio
aactaccaaatctcactttgtaaaggattcacacgatgaccactagaggtctatccgctt

SEQ ID NO: 79 - DNA, 60 bp, Danio rerio
tactaagagaagtcatgcagtgatgatttcgccgtgtacgtaccaaagtaacactgtttg

SEQ ID NO: 80 - DNA, 60 bp, Danio rerio
tgtaaaattttacagaggaaaggcaagttcactcaatagcaaattcctcatttactcacc

SEQ ID NO: 81 - DNA, 60 bp, Danio rerio
ctttaaaggcaagacactgcaggcacctgagatttcggtctttttagcttctcattcatt

SEQ ID NO: 82 - DNA, 60 bp, Danio rerio
aatggcgtatcggtaacatgatgtccagatagcttcatttcaactggaaacgatcagctg

SEQ ID NO: 83 - DNA, 60 bp, Danio rerio
gtgaactttgcatttgaaacccagcttcttagccagctcagtgagcagatccatacagta

SEQ ID NO: 84 - DNA, 60 bp, Danio rerio
atttgtgcatatgacgtatgtaacctcataaccctgaggttacgcatttgactttggcca

SEQ ID NO: 85 - DNA, 60 bp, Danio rerio
tcaaagtgaccacaactccttgcacaacattacagtgagcagtctatacaagtacatttt

SEQ ID NO: 86 - DNA, 60 bp, Danio rerio
tgtttgaggccagactttttactttcatttgagaaaaatacagtagtcagtatttcagct

SEQ ID NO: 87 - DNA, 60 bp, Danio rerio
ctttcatttagacattaatctgtcacagttctccaggcaagacgcaatgacctcagcacc

SEQ ID NO: 88 - DNA, 60 bp, Danio rerio
aattgcccactttgtatttggagaggccacaaacttgcttttttggtttgacccagtaat

SEQ ID NO: 89 - DNA, 60 bp, Danio rerio
gcgaccacacatgagctgtacagcactgttaaagaaacactccactttttatttaggaaa

SEQ ID NO: 90 - DNA, 60 bp, Danio rerio
tcattcaattgtatgctgctcgttattcagttcactggtatgttttatgtcttgcttcaa

SEQ ID NO: 91 - DNA, 60 bp, Danio rerio
ttcatgttctctgcactttaaatggcagaagaacttgtcgtttcaaccttaatgtgggtt

SEQ ID NO: 92 - DNA, 60 bp, Danio rerio
tatatgtgtgctgcaaacctgtgagatcaaatgcagtcatcctgcttcacagtttcattg

SEQ ID NO: 93 - DNA, 60 bp, artificial sequence
  ds-red control
ttcatgcgcttcaaggtgcgcatggagggctccgtgaacggccacgagttcgagatcgag

SEQ ID NO: 94 - RNA, 22 nt, Danio rerio
ugagaacugaauuccaagggug

SEQ ID NO: 95 - RNA, 23 nt, Danio rerio
uagcuuaucagacugguguuggc

SEQ ID NO: 96 - RNA, 22 nt, Danio rerio
uagcaccauuugaaaucgguua

SEQ ID NO: 97 - RNA, 22 nt, Danio rerio
accguggcauuagauuguuacu

SEQ ID NO: 98 - RNA, 22 nt, Danio rerio
uaacagucuacagccauggucg

SEQ ID NO: 99 - RNA, 23 nt, Danio rerio
ugagaacugaauuccauagaugg

SEQ ID NO: 100 - RNA, 22 nt, Danio rerio
cgacaacagucuguaggcuguc

SEQ ID NO: 101 - RNA, 22 nt, Danio rerio
uagcaccauuugaaaucagugu

SEQ ID NO: 102 - RNA, 22 nt, Danio rerio
uagguaguuucauguuguuggg

SEQ ID NO: 103 - RNA, 22 nt, Danio rerio
aauugcacgguauccaucugua

SEQ ID NO: 104 - RNA, 22 nt, Danio rerio
ggugcagugcugcaucucuggu

SEQ ID NO: 105 - RNA, 22 nt, Danio rerio
uacugcaucaggaacugauugg

SEQ ID NO: 106 - RNA, 22 nt, Danio rerio
aacuggcccgcaaagucccgcu

SEQ ID NO: 107 - RNA, 21 nt, Danio rerio
uaacagucuacagucauggcu

SEQ ID NO: 108 - RNA, 22 nt, Danio rerio
uaaugccccuaaaaauccuuau

SEQ ID NO: 109 - RNA, 22 nt, Danio rerio
uaugugcccuuggacuacaucg

SEQ ID NO: 110 - RNA, 23 nt, Danio rerio
agugacaucauauguacggcugc

SEQ ID NO: 111 - RNA, 24 nt, Danio rerio
uuuuuugcagaaacguuucagauu

SEQ ID NO: 112 - RNA, 22 nt, Danio rerio
uggcagugucuuagcugguugu